(19) United States
(12) Patent Application Publication
(10) Pub. No.: US 2021/0023065 A1
(43) Pub. Date: Jan. 28, 2021

(54) AMPHIPHILIC THIOL COMPOUNDS AND USES THEREOF

(71) Applicant: THE REGENTS OF THE UNIVERSITY OF CALIFORNIA, Oakland, CA (US)

(72) Inventors: Roberto Javier BREA FERNANDEZ, San Diego, CA (US); Andrew RUDD, La Jolla, CA (US); Neal DEVARAJ, La Jolla, CA (US); Mai JOHNSON, San Diego, CA (US); Hetika VORA, San Diego, CA (US)

(21) Appl. No.: 16/979,651

(22) PCT Filed: Mar. 12, 2019
(86) PCT No.: PCT/US19/21944

§ 371 Date: Sep. 10, 2020

Related U.S. Application Data
(60) Provisional application No. 62/641,828, filed on Mar. 12, 2018.
(60) Provisional application No. 62/740,256, filed on Oct. 2, 2018.

(51) Int. Cl. A61K 31/4402; A61K 31/426; A61K 31/105

(52) U.S. Cl. CPC A61K 31/4402; A61K 31/105; A61K 31/426

(57) ABSTRACT

Provided herein are, inter alia, amphiphilic thiol compounds and methods of using the same for the purpose of depalmitoylating proteins in cellular membranes (e.g., plasma membranes). The compounds provided herein include an amphiphilic tail, which enables them to associate with a cellular membrane and depalmitoylate (cleave native S-palmitoyl groups from) a protein in said membrane by native chemical ligation thereby triggering the protein's release from the plasma membrane. The compounds (amphiphilic thiol compounds of formula (I), (II), (III)) are, inter alia, useful for the treatment of diseases caused or associated with aberrant depalmitoylation of certain proteins (e.g., HRas, EGFR).

### CROSS-REFERENCES TO RELATED APPLICATIONS

This application claims the benefit of U.S. Provisional Application No. 62/641,828 filed Mar. 12, 2018 and U.S. Provisional Application No. 62/740,256 filed Oct. 2, 2018, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under grant no. CHE-1254611, awarded by the National Science Foundation, and grant no. LT000385/2014-C, awarded by the Human Frontier Science Program Organization. The government has certain rights in the invention.

### BACKGROUND

Post-translational S-palmitoylation plays a central role in protein localization, trafficking, stability, aggregation, and cell signaling. Dysregulation of palmitoylation pathways in cells can alter protein function and is the cause of several diseases. Considering the biological and clinical importance of S-palmitoylation, tools for direct, in vivo modulation of this lipid modification would be extremely valuable. Disclosed herein, inter alia, are solutions to these and other problems in the art.

### BRIEF SUMMARY OF THE INVENTION

In one aspect, a compound of formula:

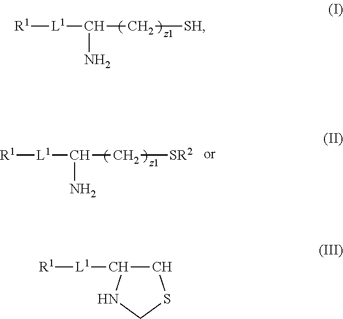

is provided.

In formula (I), (II), and (III), R¹ is hydrogen, —N(R⁴)(R⁵), —N(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is a thiol protecting group.

R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

And z1 is an integer from 0 to 5.

In another aspect, a pharmaceutical composition is provided. The pharmaceutical composition includes a pharmaceutically acceptable excipient and a compound as provided herein including embodiments thereof.

In another aspect, a method of treating a depalmitoylation-associated disease in a subject in need thereof is provided. The method includes administering to the subject a therapeutically effective amount of a compound as provided herein including embodiments thereof, thereby treating a treating a depalmitoylation-associated disease in the subject.

In another aspect, a method of depalmitoylating a protein in a cell is provided. The method includes contacting the cell with an effective amount of a compound as provided herein including embodiments thereof.

In another aspect, a method of treating a depalmitoylation-associated disease in a subject in need thereof is provided. The method includes administering to the subject a therapeutically effective amount of a depalmitoylating amphiphilic thiol compound, thereby treating a depalmitoylation-associated disease in the subject.

In another aspect, a method of treating a neurological disease in a subject in need thereof is provided. The method includes administering to the subject a therapeutically effective amount of a compound of formula:

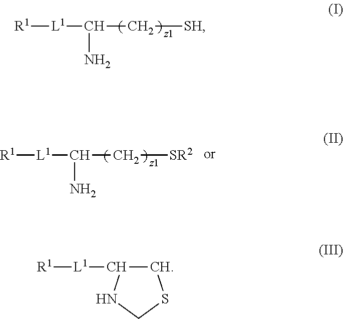

In formula (I), (II), and (III) R¹ is hydrogen, —N(R⁴)(R⁵), —N(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is a thiol protecting group. R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. And z1 is an integer from 0 to 5, thereby treating a neurological disease in the subject.

In another aspect, a method of treating cancer in a subject in need thereof is provided. The method includes administering to said subject a therapeutically effective amount of a compound of formula:

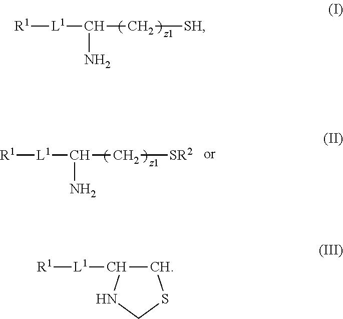

In formula (I), (II), and (III) R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is a thiol protecting group. R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. And z1 is an integer from 0 to 5, thereby treating cancer in the subject.

### BRIEF DESCRIPTION OF THE DRAWINGS

FIG. 1A-FIG. 1C. Amphiphile-mediated depalmitoylation (AMD). (FIG. 1A) Schematic representation of the depalmitoylation of an S-palmitoylated protein (SPP) by AMD. (FIG. 1B) Chemical structures of compounds 1, 2, 3, 4 are structures of the compounds provided herein. (FIG. 1C) HPLC/ELSD traces of the reaction between alkyl cysteine 1 (5 mM) and MESNA thiopalmitate 3 (5 mM) to form the N-acylated product 4.

FIG. 2A-FIG. 2F. Depalmitoylation of HRas in HeLa cells. (FIG. 2A-2F) Western blot detection of endogenous HRas in acyl resin-assisted capture fractions. The input fraction contains all cellular proteins. The palmitoylated fraction contains only S-palmitoylated proteins. Assays were performed in three biological replicates with the vehicle or control normalized to 1. Values are shown as means±SD. Statistically significant differences in palmitoylation between TCEP only (FIG. 2D) or PB (FIG. 2F) and the other means are indicated: **P<0.01, ****P<0.0001. ns, not significant.

FIG. 3A-FIG. 3E. AMD induces translocation of EGFP-HRas from the plasma membrane (PM). Fluorescence microscopy images of HeLa cells expressing EGFP-HRas (FIG. 3A-FIG. 3C) or EGFP-KRas4b (FIG. 3D) before and after treatment with 1 (FIG. 3A and FIG. 3D), 2 (FIG. 3B) or TCEP only (FIG. 3C). (FIG. 3E) Change in PM localization of EGFP-HRas or EGFP-KRas4b within large populations of cells after treatment with 1, 2, or TCEP only. The difference between the percentage of cells showing EGFP localization at the plasma membrane after (% Ca) and before (% Cb) treatment is shown. Values are reported as means±SD. Statistically significant changes in protein localization after treatment are indicated: ****P<0.0001.

FIG. 4A-FIG. 4C. Depalmitoylation of GAP43 in INCL fibroblasts. (FIG. 4A) Viability of INCL lymphoblasts after treatment with 1 for 24 h as determined by a WST-1 assay. (FIG. 4B, FIG. 4C) Western blot detection of endogenous GAP43 in acyl resin-assisted capture fractions. The input fraction contains all cellular proteins. The palmitoylated fraction contains only S-palmitoylated proteins. Assays were performed in three biological replicates with the vehicle normalized to 1. Values are shown as means±SD. Statistically significant differences in palmitoylation between TCEP only and the other means is indicated: **P<0.01. ns, not significant

FIG. 5A-FIG. 5B. Synthesis of H₂N-L-Cys-Oct (1) and H₂N-L-Ser-Oct (2).

FIG. 6. Synthesis of MESNA thiopalmitate (3).

FIG. 7A-FIG. 7B. (FIG. 7A) HPLC traces of the reaction between 1 and 3 in vitro to form 4. (FIG. 7B) HPLC traces show no reaction between 2 and 3. MS (ESI) spectra were integrated for each peak to confirm compound identity. Calculated and observed major MS peaks are listed next to corresponding ELSD peaks.

FIG. 8. HPLC/ELSD traces of a 5 mM solution of 1′ in the absence (top) and presence (bottom) of TCEP (5 mM), showing the reduction of disulfide (1′, RS SR) to thiol (1, RSH).

FIG. 9A-FIG. 9C. LC MS/MS traces of selected reaction monitoring for the AMD product 4 in HeLa cell lipid extracts. (FIG. 9A) Standard injection of 4. (FIG. 9B) Lipid extract from HeLa cells treated with TCEP control. (FIG. 9C) Lipid extract from HeLa cells treated with 1 and TCEP. Peaks corresponding to 4 are labeled with corresponding retention time (RT).

FIG. 10A-FIG. 10B. Depalmitoylation of proteins in HeLa cells. (FIG. 10A) Western blot detection of endogenous S-palmitoylated proteins in acyl resin-assisted capture fractions after treatment with vehicle or 1 (1.25 μmol/10⁷ cells). The input fraction (IF) contains all cellular proteins. The palmitoylated fraction (PF) contains only S-palmitoylated proteins. Assays were performed in three biological replicates. (FIG. 10B) Quantification of protein palmitoylation in cells treated with 1 (1.25 μmol/10⁷ cells) relative to cells treated with vehicle. Values are shown as means±SD.

FIG. 11A-FIG. 11H. Depalmitoylation of HRas in HeLa cells. Fluorescence microscopy images and intensity profiles [along white dashed lines] of HeLa cells expressing EGFP-HRas (FIG. 11A-FIG. 11F) or EGFP-KRas (FIG. 11G-FIG. 11H) before and after treatment with 1 (FIG. 11A, FIG. 11B, FIG. 11G and FIG. 11H) (1.25 μmol/10⁷ cells), 2 (C and D) (1.25 μmol/10⁷ cells) or TCEP only (FIG. 11E and FIG. 11F).

FIG. 12. Depalmitoylation of HRas in HeLa cells. Representative fluorescence microscopy images of HeLa cells expressing EGFP-HRas or EGFP-KRas before and after treatment with 1 (1.25 μmol/10⁷ cells), 2 (1.25 μmol/10⁷ cells) or TCEP only.

FIG. 13A-FIG. 13B. AKT1 and ERK1/2 phosphorylation after AMD in HeLa cells. (FIG. 13A) Western blot detection of phosphorylated AKT1 (pS473) and ERK1/2 (pY204/187) after treatment with Vehicle or 1 (1.25 μmol/10⁷ cells) and stimulation with EGF (100 ng/μL). Rab11 was used as a loading control. (FIG. 13B) Assays were performed in three biological replicates. The intensity of the protein band in each condition was normalized to the intensity of the corresponding loading control (Rab11). The values within individual replicates were normalized to the vehicle condition and reported as means±SD. ns, not significant, **P<0.01.

FIG. 14A-FIG. 14B. AKT1 and ERK1/2 phosphorylation after AMD in T24 cells. (FIG. 14A) Western blot detection of phosphorylated AKT1 (pS473) and ERK1/2 (pY204/187) after treatment with Vehicle or 1 (1.25 μmol/10⁷ cells). Rab11 was used as a loading control. (FIG. 14B) Assays were performed in three biological replicates. The intensity of the protein band in each condition was normalized to the intensity of the corresponding loading control (Rab11). The values within individual replicates were normalized to the vehicle condition and reported as means±SD.

FIG. 15. The FIG. shows synthesis and derivatization of peptides and proteins using native chemical ligation (NCL) (Janssen et al, 2015).

FIG. 16. The FIG. shows construction of large nucleic acids using native chemical ligation (NCL) (Mattes et al, 2001).

FIG. 17. The FIG. shows the mechanism of native chemical ligation (NCL). The mechanism involves a two-step process consisting of a thiol-exchange step between a C-terminal acyl thioester and the sulfhydryl moiety of an N-terminal cysteine residue in a lysolipid, which prompts an intramolecular nucleophilic attack by the α-amino group of the cysteine (S→N) acyl rearrangement) 5 to form the final amide bond.

FIG. 18. The FIG. shows a schematic representation of the depalmitoylation of an S-palmitoylated protein (SPP) by depalmitoylation through native chemical ligation (dNCL).

FIG. 19. The FIG. shows depalmitoylation of H-Ras in HeLa cells. Fluorescence microscopy images of HeLa cells expressing H-Ras-GFP before and after treatment with octyl cysteine (depalmitoylating agent).

FIG. 20. The FIG. shows a photocaged depalmitoylating agent for depalmitoylation through native chemical ligation (dNCL).

FIG. 21. The FIG. shows cell viability (WST-1) in HeLa cells.

FIG. 22. The FIG. shows a reaction scheme for prodrug cleavage by endogenous esterases.

FIG. 23. The FIG. shows synthesis of a prodrug.

FIG. 24. The FIG. shows alkyl cysteine prodrugs and alkyl cysteine derivatives.

FIG. 25. The FIG. shows HeLa cells 0 minutes and 15 minutes after administration of 20 μM S-methyl acetate alkyl cysteine.

FIG. 26. Quantification of protein palmitoylation by acyl resin-assisted capture in cells treated with alkyl cysteine (1.25 μmol/10⁷ cells) relative to cells treated with vehicle. Values are shown as means±SD.

FIG. 27A-FIG. 27B. AKT1 and ERK1/2 phosphorylation after AMD in T24 cells. (FIG. 27A) Western blot detection of phosphorylated AKT1 (pS473) and ERK1/2 (pY204/187) after treatment with Vehicle or alkyl cysteine (1.25 μmol/10⁷ cells). Rab11 was used as a loading control. (FIG. 27B) Assays were performed in three biological replicates. The intensity of the protein band in each condition was normalized to the intensity of the corresponding loading control (Rab11). The values within individual replicates were normalized to the vehicle condition and reported as means±SD.

FIG. 28. Chemical formulae of exemplary compounds are shown.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of 0, N, and S where the ring is saturated or unsaturated, but not aromatic. The 3 or 4 membered ring contains 1 heteroatom selected from the group consisting of O, N and S. The κ membered ring can contain zero or one double bond and one, two or three heteroatoms selected from the group consisting of O, N and S. The κ or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

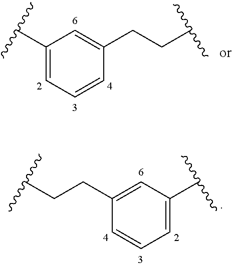 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, FIG.s, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)-for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
   (l) metal silicon oxide bonding; and
   (m) metal bonding to reactive phosphorus groups (e.g. phosphines) to form, for example, phosphate diester bonds.
   (n) azides coupled to alkynes using copper catalyzed cycloaddition click chemistry.
   (o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-Cao alkyl, or unsubstituted 2 to 20 membered heteroalkyl,” the group may contain one or more unsubstituted C₁-Cao alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁷⁷Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴E, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴E, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A “thiol protecting group” or “disulfide protecting group” as used herein refers in accordance with its ordinary meaning in organic chemistry to a moiety covalently bound to a sulphur of a thiol moiety (e.g., a sulphur of a heteroalkyl, a sulphur of a heterocycloalkyl, or a sulphur of a heteroaryl) to prevent unspecific reactivity of the sulphur of thiol moiety (e.g., a sulphur of a heteroalkyl, a sulphur of a heterocycloalkyl, or a sulphur of a heteroaryl) in solution in vitro or in vivo or reactivity during one or more chemical reactions performed prior to removal of the protecting group. For the invention provided herein the thiol protecting group prevents oxidation of the thiol and may improve its bioavailability and cellular delivery. Upon entering the cell, the thiol protecting group is cleaved and the free thiol is exposed, forming an active depalmitoylating amphiphilic thiol compound provided herein including embodiments thereof. Typically a protecting group is bound to a heteroatom (e.g., S) during a part of a multipart synthesis wherein it is not desired to have the heteroatom react (e.g., a chemical reduction) with the reagent. Alternatively, the thiol protecting group prevents the sulphur to react with reactants other than the intended target protein (e.g., a protein requiring depalmitoylation and forming part of a plasma membrane). Following protection the protecting group may be removed (e.g., by modulating the pH). Any one of the protecting moieties described in Vrudhula et al. (Vrudhula V. M.; Macmaster, J. F.; Li, Z.; Kerr, E.; Senter, P. D. Reductively Activated Disulfide Prodrugs of Paclitaxel. Bioorganic Med. Chem. Lett. 2002, 12, 3591-3594) and Fan et al. (Fan, W.; Wu, Y.; Li, X.; Yao, N.; Li, X.; Yu, Y.; Hai, L. Design, Synthesis and Biological Evaluation of Brain-Specific Glucosyl Thiamine Disulfide Prodrugs of Naproxen. Eur. J. Med. Chem. 2011, 46, 3651-3661), which are incorporated by reference herein in their entirety and for all purposes, may be used as thiol protecting groups for the compositors and methods provided herein.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “EGFP” or “EGFP protein” as used herein refers to any of the recombinant or naturally-occurring forms of the Enhanced Green Fluorescent Protein (EGFP), also known as enhanced GFP, or variants or homologs thereof that maintain EGFP activity (e.g., within at least 50%, 80%, 90%, 95%, 96%, 97%, 98%, 99% or 100% activity compared to EGFP). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring EGFP protein. In embodiments, the EGFP protein is substantially identical to the protein identified by the NCBI reference number GI: 13194618, or a variant or homolog having substantial identity thereto. In embodiments, the EGFP protein is substantially identical to the protein identified by the NCBI reference number GI: 1373316, or a variant or homolog having substantial identity thereto. In embodiments, the EGFP protein is substantially identical to the protein identified by the NCBI reference number GI: 669204078, or a variant or homolog having substantial identity thereto. In embodiments, the EGFP protein is substantially identical to the protein identified by the NCBI reference number GI: 1373319, or a variant or homolog having substantial identity thereto.

The term “HRas” or “HRas protein” as used herein refers to any of the recombinant or naturally-occurring forms of the GTPase HRas, also known as transforming protein p21, or variants or homologs thereof that maintain HRas activity (e.g., within at least 50%, 80%, 90%, 95%, 96%, 97%, 98%, 99% or 100% activity compared to HRas). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring HRas protein. In embodiments, the HRas protein is substantially identical to the protein identified by the NCBI reference number GI:4885425, or a variant or homolog having substantial identity thereto. In embodiments, the HRas protein is substantially identical to the protein identified by the NCBI reference number GI:34222246, or a variant or homolog having substantial identity thereto. In embodiments, the HRas protein is substantially identical to the protein identified by the NCBI reference number GI:194363762, or a variant or homolog having substantial identity thereto. In embodiments, the HRas protein is substantially identical to the protein identified by the NCBI reference number GI:968121903, or a variant or homolog having substantial identity thereto.

The term “NRas” or “NRas protein” as used herein refers to any of the recombinant or naturally-occurring forms of the GTPase NRas, or variants or homologs thereof that maintain NRas activity (e.g., within at least 50%, 80%, 90%, 95%, 96%, 97%, 98%, 99% or 100% activity compared to NRas). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring NRas protein. In embodiments, the NRas protein is substantially identical to the protein identified by the NCBI reference number GI: 4505451, or a variant or homolog having substantial identity thereto.

The term “EGFR” or “EGFR protein” as used herein refers to any of the recombinant or naturally-occurring forms of the Epidermal Growth Factor Receptor (EGFR) tyrosine kinase, also known as epidermal growth factor receptor, ErbB-1 or HER1 in humans, or variants or homologs thereof that maintain EGFR activity (e.g., within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring EGFR protein. In embodiments, the EGFR protein is substantially identical to the protein identified by the NCBI reference number GI: 1101020101, or a variant or homolog having substantial identity thereto. In embodiments, the EGFR protein is substantially identical to the protein identified by the NCBI reference number GI: 1100832916, or a variant or homolog having substantial identity thereto. In embodiments, the EGFR protein is substantially identical to the protein identified by the NCBI reference number GI: 1100818978, or a variant or homolog having substantial identity thereto. In embodiments, the EGFR protein is substantially identical to the protein identified by the NCBI reference number GI: 1100818972, or a variant or homolog having substantial identity thereto.

The term “amyloid precursor protein” as used herein refers to any of the recombinant or naturally-occurring forms of the amyloid precursor protein, also known as APP, or variants or homologs thereof that maintain amyloid precursor protein activity (e.g., within at least 50%, 80%, 90%, 95%, 96%, 97%, 98%, 99% or 100% activity compared to amyloid precursor protein). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring amyloid precursor protein. In embodiments, the amyloid precursor protein is substantially identical to the protein identified by the NCBI reference number GI: 4502167, or a variant or homolog having substantial identity thereto. In embodiments, the amyloid precursor protein is substantially identical to the protein identified by the NCBI reference number GI: 41406055, or a variant or homolog having substantial identity thereto. In embodiments, the amyloid precursor protein is substantially identical to the protein identified by the NCBI reference number GI: 41406057, or a variant or homolog having substantial identity thereto. In embodiments, the amyloid precursor protein is substantially identical to the protein identified by the NCBI reference number GI: 209862833, or a variant or homolog having substantial identity thereto.

The term “BACE1” or “BACE1 protein” as used herein refers to any of the recombinant or naturally-occurring forms of the Beta-secretase 1 (BACE1) aspartic-acid protease, also known as beta-site amyloid precursor protein cleaving enzyme 1, beta-site APP cleaving enzyme 1, membrane-associated aspartic protease 2, memapsin-2, aspartyl protease 2, and ASP2, or variants or homologs thereof that maintain BACE1 activity (e.g., within at least 50%, 80%, 90%, 95%, 96%, 97%, 98%, 99% or 100% activity compared to BACE1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BACE1 protein. In embodiments, the BACE1 protein is substantially identical to the protein identified by the NCBI reference number GI: 6912266, or a variant or homolog having substantial identity thereto. In embodiments, the BACE1 protein is substantially identical to the protein identified by the NCBI reference number GI: 21040364, or a variant or homolog having substantial identity thereto. In embodiments, the BACE1 protein is substantially identical to the protein identified by the NCBI reference number GI: 21040366, or a variant or homolog having substantial identity thereto. In embodiments, the BACE1 protein is substantially identical to the protein identified by the NCBI reference number GI: 21040368, or a variant or homolog having substantial identity thereto.

The term “EZH2” or “EZH2 protein” as used herein refers to any of the recombinant or naturally-occurring forms of the Enhancer of Zeste Homolog 2 (EZH2) histone-lysine N-methyltransferase, or variants or homologs thereof that maintain EZH2 activity (e.g., within at least 50%, 80%, 90%, 95%, 96%, 97%, 98%, 99% or 100% activity compared to EZH2). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring EZH2 protein. In embodiments, the EZH2 protein is substantially identical to the protein identified by the NCBI reference number GI: 21361095, or a variant or homolog having substantial identity thereto. In embodiments, the EZH2 protein is substantially identical to the protein identified by the NCBI reference number GI: 23510384, or a variant or homolog having substantial identity thereto. In embodiments, the EZH2 protein is substantially identical to the protein identified by the NCBI reference number GI: 322506097, or a variant or homolog having substantial identity thereto. In embodiments, the EZH2 protein is substantially identical to the protein identified by the NCBI reference number GI: 322506099, or a variant or homolog having substantial identity thereto.

The term “PD-L1” or “PD-L1 protein” as used herein refers to any of the recombinant or naturally-occurring forms of Programmed Death Ligand 1 (PD-L1), also known as cluster of differentiation 274 (CD274) or B7 homolog 1 (B7-H1), or variants or homologs thereof that maintain PD-L1 activity (e.g., within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring PD-L1 protein. In embodiments, the PD-L1 protein is substantially identical to the protein identified by the NCBI reference number GI: 7661534, or a variant or homolog having substantial identity thereto. In embodiments, the PD-L1 protein is substantially identical to the protein identified by the NCBI reference number GI: 390979639, or a variant or homolog having substantial identity thereto. In embodiments, the PD-L1 protein is substantially identical to the protein identified by the NCBI reference number GI: 930425329, or a variant or homolog having substantial identity thereto.

The term “flotillin-1” or “flotillin-1 protein” as used herein refers to any of the recombinant or naturally-occurring forms of flotillin-1, or variants or homologs thereof that maintain flotillin-1 activity (e.g., within at least 50%, 80%, 90%, 95%, 96%, 97%, 98%, 99% or 100% activity compared to flotillin-1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring flotillin-1 protein. In embodiments, the flotillin-1 protein is substantially identical to the protein identified by the NCBI reference number GI: 5031699, or a variant or homolog having substantial identity thereto. In embodiments, the flotillin-1 protein is substantially identical to the protein identified by the NCBI reference number GI: 974141105, or a variant or homolog having substantial identity thereto.

The term “flotillin-2” or “flotillin-2 protein” as used herein refers to any of the recombinant or naturally-occurring forms of flotillin-2, or variants or homologs thereof that maintain flotillin-2 activity (e.g., within at least 50%, 80%, 90%, 95%, 96%, 97%, 98%, 99% or 100% activity compared to flotillin-2). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring flotillin-2 protein. In embodiments, the flotillin-2 protein is substantially identical to the protein identified by the NCBI reference number GI: 94538362, or a variant or homolog having substantial identity thereto.

The term “calnexin” or “calnexin protein” as used herein refers to any of the recombinant or naturally-occurring forms of calnexin, or variants or homologs thereof that maintain calnexin activity (e.g., within at least 50%, 80%, 90%, 95%, 96%, 97%, 98%, 99% or 100% activity compared to calnexin). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring calnexin protein. In embodiments, the calnexin protein is substantially identical to the protein identified by the NCBI reference number GI: 10716563, or a variant or homolog having substantial identity thereto. In embodiments, the calnexin protein is substantially identical to the protein identified by the NCBI reference number GI: 66933005, or a variant or homolog having substantial identity thereto. In embodiments, the calnexin protein is substantially identical to the protein identified by the NCBI reference number GI: 1395168545, or a variant or homolog having substantial identity thereto. In embodiments, the calnexin protein is substantially identical to the protein identified by the NCBI reference number GI: 1395168466, or a variant or homolog having substantial identity thereto.

The term “Gα(i)” or “Gα(i) protein” as used herein refers to any of the recombinant or naturally-occurring forms of Gᵢ alpha subunit (Gα(i)), also known as Gᵢ/G₀ or Gᵢ protein, or variants or homologs thereof that maintain Gα(i) activity (e.g., within at least 50%, 80%, 90%, 95%, 96%, 97%, 98%, 99% or 100% activity compared to Gα(i)). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Gα(i) protein. In embodiments, the Gα(i) protein is substantially identical to the protein identified by the NCBI reference number GI: 33946324, or a variant or homolog having substantial identity thereto. In embodiments, the Gα(i) protein is substantially identical to the protein identified by the NCBI reference number GI: 374081863, or a variant or homolog having substantial identity thereto.

The term “metadherin” or “metadherin protein” as used herein refers to any of the recombinant or naturally-occurring forms of metadherin, also known as protein LYRIC or astrocyte elevated gene-1 protein (AEG-1), or variants or homologs thereof that maintain metadherin activity (e.g., within at least 50%, 80%, 90%, 95%, 96%, 97%, 98%, 99% or 100% activity compared to metadherin). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring metadherin protein. In embodiments, the metadherin protein is substantially identical to the protein identified by the NCBI reference number GI: 223555917, or a variant or homolog having substantial identity thereto. In embodiments, the metadherin protein is substantially identical to the protein identified by the NCBI reference number GI: 1034661969, or a variant or homolog having substantial identity thereto.

The term “CD44” or “CD44 protein” as used herein refers to any of the recombinant or naturally-occurring forms of Cluster of Differentiation 44 (CD44), also known as HCAM (homing cell adhesion molecule), Pgp-1 (phagocytic glycoprotein-1), Hermes antigen, lymphocyte homing receptor, ECM-III, and HUTCH-1, or variants or homologs thereof that maintain CD44 activity (e.g., within at least 50%, 80%, 90%, 95%, 96%, 97%, 98%, 99% or 100% activity compared to CD44). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring CD44 protein. In embodiments, the CD44 protein is substantially identical to the protein identified by the NCBI reference number GI: 48255941, or a variant or homolog having substantial identity thereto. In embodiments, the CD44 protein is substantially identical to the protein identified by the NCBI reference number GI: 48255935, or a variant or homolog having substantial identity thereto. In embodiments, the CD44 protein is substantially identical to the protein identified by the NCBI reference number GI: 48255937, or a variant or homolog having substantial identity thereto. In embodiments, the CD44 protein is substantially identical to the protein identified by the NCBI reference number GI: 321400138, or a variant or homolog having substantial identity thereto.

The term “SNAP25” or “SNAP25 protein” as used herein refers to any of the recombinant or naturally-occurring forms of Synaptosomal Nerve-Associated Protein 25 (SNAP25), or variants or homologs thereof that maintain SNAP25 activity (e.g., within at least 50%, 80%, 90%, 95%, 96%, 97%, 98%, 99% or 100% activity compared to SNAP25). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring SNAP25 protein. In embodiments, the SNAP25 protein is substantially identical to the protein identified by the NCBI reference number GI: 18765735, or a variant or homolog having substantial identity thereto. In embodiments, the SNAP25 protein is substantially identical to the protein identified by the NCBI reference number GI: 18765733, or a variant or homolog having substantial identity thereto. In embodiments, the SNAP25 protein is substantially identical to the protein identified by the NCBI reference number GI: 1018443229, or a variant or homolog having substantial identity thereto. In embodiments, the SNAP25 protein is substantially identical to the protein identified by the NCBI reference number GI: 1018443211, or a variant or homolog having substantial identity thereto.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The term “depalmitoylation-associated disease” is used to broadly refer to disorders or symptoms of diseases associated with a level of depalmitoylation of a protein. In embodiments, the disease is caused by, or a symptom of the disease is caused by aberrant depalmitoylation (e.g., less or more compared to a standard control).

The term “associated” or “associated with” in the context of a level of protein modification (e.g., depalmitoylation) or substance activity (e.g., depalmitoylation activity) associated with a disease means that the disease is caused by (in whole or in part), or a symptom of the disease is caused by (in whole or in part) the level of protein modification or substance activity or function (i.e., depalmitoylation, depalmitoylation activity). For example, a disease associated with depalmitoylation or a symptom of an depalmitoylation-associated disease or condition associated with an increase or decrease in depalmitoylation activity may be a disease or symptom that results (entirely or partially) from an increase or decrease in depalmitoylation activity (e.g. increase or decrease in depalmitoylation of a protein).

Non-limiting examples of depalmitoylation-associated diseases include cancer and neurodegenerative diseases, e.g., bladder cancer, head and neck cancer, Costello's Syndrome, melanoma, acute myeloid lymphoma (AML), non-small cell lung carcinoma, Alzheimer's disease, infantile neuronal ceroid lipofuscinosis or glioma.

A “standard control” as referred to herein refers to a sample that serves as a reference, usually a known reference, for comparison to a test sample. For example, a test sample can be taken from a patient suspected of having a depalmitoylation-associated disease (e.g., cancer) and compared to samples from a known depalmitoylation-associated disease (e.g., cancer) patient, or a known normal (non-disease) individual. A control can also represent an average value gathered from a population of similar individuals, e.g., depalmitoylation-associated disease (e.g., cancer) patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In some examples of the disclosed methods, when the level of depalmitoylation of a protein (e.g., HRas) is assessed, the level is compared with a control level of depalmitoylation of the same or a different protein. By control level is meant the level of depalmitoylation from a sample or subject lacking a depalmitoylation-associated disease (e.g., cancer), a sample or subject at a selected stage of a depalmitoylation-associated disease (e.g., cancer), or in the absence of a particular variable such as a therapeutic agent (e.g., chemotherapeutic agent). Alternatively, the control level comprises a known amount of depalmitoylation of the protein Such a known amount correlates with an average level of subjects lacking the depalmitoylation-associated disease (e.g., cancer), at a selected stage of the depalmitoylation-associated disease (e.g., cancer), or in the absence of a particular variable such as a therapeutic agent. A control level also includes the level of depalmitoylation of a protein from one or more selected samples or subjects as described herein. For example, a control level includes an assessment of the level of depalmitoylation of a protein in a sample from a subject that does not have a depalmitoylation-associated disease (e.g., cancer), is not at a selected stage of a depalmitoylation-associated disease (e.g., cancer), or has not received treatment for a depalmitoylation-associated disease (e.g., cancer). Another exemplary control level includes an assessment of the level of depalmitoylation of a protein in samples taken from multiple subjects that do not have a depalmitoylation-associated disease (e.g., cancer), are at a selected stage of a depalmitoylation-associated disease (e.g., cancer), or have not received treatment for a depalmitoylation-associated disease (e.g., cancer).

When the control level includes the level of depalmitoylation of a protein in a sample or subject in the absence of a chemotherapeutic agent, the control sample or subject is optionally the same sample or subject to be tested before or after treatment with a chemotherapeutic agent or is a selected sample or subject in the absence of the therapeutic agent. Alternatively, a control level is an average level calculated from a number of subjects without a particular disease. A control level also includes a known control level or value known in the art.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-compound interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Compounds

Provided herein are, inter alia, amphiphilic thiol compounds and methods of using the same for the purpose of depalmitoylating proteins in cellular membranes (plasma membrane). The compounds provided herein include an amphiphilic tail, which enables them to associate with a cellular membrane and depalmitoylate (cleave native S-palmitoyl groups from) a protein in said membrane by native chemical ligation thereby triggering the protein's release from the plasma membrane. The compounds (amphiphilic thiol compounds of formula (I), (II), (III)) are, inter alia, useful for the treatment of diseases caused or associated with aberrant depalmitoylation of certain proteins (e.g., HRas, EGFR).

In one aspect, a compound of formula:

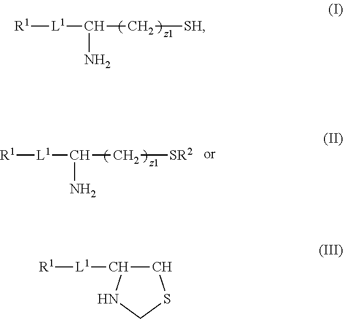

is provided.

In formula (I), (II), and (III), R¹ is hydrogen, —N(R⁴)(R⁵), —N(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is a thiol protecting group.

R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

And z1 is an integer from 0 to 5.

In embodiments, the compound has the formula:

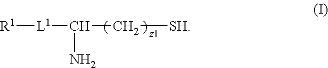

In embodiments, the compound has the formula:

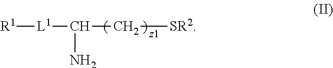

In embodiments, the compound has the formula:

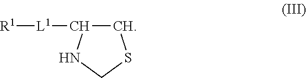

In formula (I), (II) and (III), R¹ may be —N(R⁴)(R⁵), —N⁺(R⁴)(R⁵)(R⁶), substituted or unsubstituted C₁-C₂₅ alkyl, or substituted or unsubstituted aryl. In embodiments, R¹ is —N(R⁴)(R⁵). In embodiments, R¹ is —N(R⁴)(R⁵)(R⁶). In embodiments, R¹ is substituted or unsubstituted C₁-C₂₅ alkyl. In embodiments, R¹ is substituted C₁-C₂₅ alkyl. In embodiments, R¹ is unsubstituted C₁-C₂₅ alkyl. In embodiments, R¹ is substituted or unsubstituted aryl. In embodiments, R¹ is substituted aryl. In embodiments, R¹ is unsubstituted aryl.

In embodiments, R¹ is —N(R⁴)(R⁵) and R⁴ and R⁵ are independently unsubstituted C₁-C₁₀ alkyl. In embodiments, R⁴ and R⁵ are independently unsubstituted C₂-C₁₀ alkyl. In embodiments, R⁴ and R⁵ are independently unsubstituted C₃-C₁₀ alkyl. In embodiments, R⁴ and R⁵ are independently unsubstituted C₄-C₁₀ alkyl. In embodiments, R⁴ and R⁵ are independently unsubstituted C₅-C₁₀ alkyl. In embodiments, R⁴ and R⁵ are independently unsubstituted C₆-C₁₀ alkyl. In embodiments, R⁴ and R⁵ are independently unsubstituted C₇-C₁₀ alkyl. In embodiments, R⁴ and R⁵ are independently unsubstituted C₈-C₁₀ alkyl. In embodiments, R⁴ and R⁵ are independently unsubstituted C₁-C₉ alkyl. In embodiments, R⁴ and R⁵ are independently unsubstituted C₁-C₈ alkyl. In embodiments, R⁴ and R⁵ are independently unsubstituted C₁-C₇ alkyl. In embodiments, R⁴ and R⁵ are independently unsubstituted C₁-C₆ alkyl. In embodiments, R⁴ and R⁵ are independently unsubstituted C₁-C₅ alkyl. In embodiments, R⁴ and R⁵ are independently unsubstituted C₁-C₄ alkyl. In embodiments, R⁴ and R⁵ are independently unsubstituted C₁-C₃ alkyl. In embodiments, R⁴ and R⁵ are independently unsubstituted C₁, C₂, C₃, C₄, C₅, C₆, C₇, C₈, C₉, or C₁₀ alkyl. In embodiments, R⁴ and R⁵ are independently unsubstituted C₁ alkyl.

In embodiments, R¹ is —N(R⁴)(R⁵)(R⁶) and R⁴, R⁵ and R⁶ are independently unsubstituted C₁-C₁₀ alkyl. In embodiments, R⁴, R⁵ and R⁶ are independently unsubstituted C₂-C₁₀ alkyl. In embodiments, R⁴, R⁵ and R⁶ are independently unsubstituted C₃-C₁₀ alkyl. In embodiments, R⁴, R⁵ and R⁶ are independently unsubstituted C₄-C₁₀ alkyl. In embodiments, R⁴, R⁵ and R⁶ are independently unsubstituted C₅-C₁₀ alkyl. In embodiments, R⁴, R⁵ and R⁶ are independently unsubstituted C₆-C₁₀ alkyl. In embodiments, R⁴, R⁵ and R⁶ are independently unsubstituted C₇-C₁₀ alkyl. In embodiments, R⁴, R⁵ and R⁶ are independently unsubstituted C₈-C₁₀ alkyl. In embodiments, R⁴, R⁵ and R⁶ are independently unsubstituted C₁-C₉ alkyl. In embodiments, R⁴, R⁵ and R⁶ are independently unsubstituted C₁-C₈ alkyl. In embodiments, R⁴, R⁵ and R⁶ are independently unsubstituted C₁-C₇ alkyl. In embodiments, R⁴, R⁵ and R⁶ are independently unsubstituted C₁-C₆ alkyl. In embodiments, R⁴, R⁵ and R⁶ are independently unsubstituted C₁-C₅ alkyl. In embodiments, R⁴, R⁵ and R⁶ are independently unsubstituted C₁-C₄ alkyl. In embodiments, R⁴, R⁵ and R⁶ are independently unsubstituted C₁-C₃ alkyl. In embodiments, R⁴, R⁵ and R⁶ are independently unsubstituted C₁ alkyl, C₂ alkyl, C₃ alkyl, C₄ alkyl, C₅ alkyl, C₆ alkyl, C₇ alkyl, C₈ alkyl, C₉ alkyl or C₁₀ alkyl. In embodiments, R⁴, R⁵ and R⁶ are independently unsubstituted C₁ alkyl.

In embodiments, R¹ is unsubstituted C₁-C₂₅ alkyl. In embodiments, R¹ is unsubstituted C₂-C₂₅ alkyl. In embodiments, R¹ is unsubstituted C₃-C₂₅ alkyl. In embodiments, R¹ is unsubstituted C₄-C₂₅ alkyl. In embodiments, R¹ is unsubstituted C₅-C₂₅ alkyl. In embodiments, R¹ is unsubstituted C₆-C₂₅ alkyl. In embodiments, R¹ is unsubstituted C₇-C₂₅ alkyl. In embodiments, R¹ is unsubstituted C₈-C₂₅ alkyl. In embodiments, R¹ is unsubstituted C₉-C₂₅ alkyl. In embodiments, R¹ is unsubstituted C₁₀-C₂₅ alkyl. In embodiments, R¹ is unsubstituted C₁₁-C₂₅ alkyl. In embodiments, R¹ is unsubstituted C₁₂-C₂₅ alkyl. In embodiments, R¹ is unsubstituted C₁₃-C₂₅ alkyl. In embodiments, R¹ is unsubstituted C₁₄-C₂₅ alkyl. In embodiments, R¹ is unsubstituted C₁₅-C₂₅ alkyl. In embodiments, R¹ is unsubstituted C₁₆-C₂₅ alkyl. In embodiments, R¹ is unsubstituted C₁₁-C₂₅ alkyl. In embodiments, R¹ is unsubstituted C₁₈-C₂₅ alkyl. In embodiments, R¹ is unsubstituted C₁₉-C₂₅ alkyl. In embodiments, R¹ is unsubstituted C₂₀-C₂₅ alkyl. In embodiments, R¹ is unsubstituted C₂₁-C₂₅ alkyl. In embodiments, R¹ is unsubstituted C₂₂-C₂₅ alkyl. In embodiments, R¹ is unsubstituted C₂₃-C₂₅ alkyl.

In embodiments, R¹ is unsubstituted C₁-C₂₄ alkyl. In embodiments, R¹ is unsubstituted C₁-C₂₃ alkyl. In embodiments, R¹ is unsubstituted C₁-C₂₂ alkyl. In embodiments, R¹ is unsubstituted C₁-C₂₁ alkyl. In embodiments, R¹ is unsubstituted C₁-C₂₀ alkyl. In embodiments, R¹ is unsubstituted C₁-C₁₉ alkyl. In embodiments, R¹ is unsubstituted C₁-C₁₈ alkyl. In embodiments, R¹ is unsubstituted C₁-C₁₇ alkyl. In embodiments, R¹ is unsubstituted C₁-C₁₆ alkyl. In embodiments, R¹ is unsubstituted C₁-C₁₅ alkyl. In embodiments, R¹ is unsubstituted C₁-C₁₄ alkyl. In embodiments, R¹ is unsubstituted C₁-C₁₃ alkyl. In embodiments, R¹ is unsubstituted C₁-C₁₂ alkyl. In embodiments, R¹ is unsubstituted C₁-C₁₁ alkyl. In embodiments, R¹ is unsubstituted C₁-C₁₀ alkyl. In embodiments, R¹ is unsubstituted C₁-C₉ alkyl. In embodiments, R¹ is unsubstituted C₁-C₈ alkyl. In embodiments, R¹ is unsubstituted C₁-C₇ alkyl. In embodiments, R¹ is unsubstituted C₁-C₆ alkyl. In embodiments, R¹ is unsubstituted C₁-C₅ alkyl. In embodiments, R¹ is unsubstituted C₁-C₄ alkyl. In embodiments, R¹ is unsubstituted C₁-C₃ alkyl. In embodiments, R¹ is unsubstituted C₂₅ alkyl, C₂₄ alkyl, C₂₃ alkyl, C₂₂ alkyl, C₂₁ alkyl, C₂₀ alkyl, C₁₉ alkyl, C₁₈ alkyl, C₁₇ alkyl, C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₆ alkyl, C₅ alkyl, C₄ alkyl, C₃ alkyl, C₂ alley or C₁ alkyl.

In embodiments, R¹ is unsubstituted C₈ alkyl.

In embodiments, R¹ is hydrogen, —N(R⁴)(R⁵), —N(R⁴)(R⁵)(R⁶), substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R¹ is hydrogen, —N(R⁴)(R⁵), —N(R⁴)(R⁵)(R⁶), substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹ may be R^{1A}-substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl.

R^{1A} is hydrogen, —N(R^{4A})(R^{5A}), —N⁺(R^{4A})(R^{5A})(R^{6A}), substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R^{1A} is hydrogen, —N(R^{4A})(R^{5A}), —N⁺(R^{4A})(R^{5A})(R^{6A}), substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A} may be R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl.

R^{1B} is hydrogen, —N(R^{4B})(R^{5B}), —N⁺(R^{4B})(R^{5AB})(R^{6B}), substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R^{1B} is hydrogen, —N(R^{4B})(R^{5B}), —N(R^{4B})(R^{5B})(R^{6B}), substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1B} may be R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl.

R^{1C} is hydrogen, —NH₂, —N⁺H₃, unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1C} is hydrogen, —NH₂, —N⁺H₃, unsubstituted alkyl (e.g., C₁-C₈, C₁-C₆, or C₁-C₄), unsubstituted heteroalkyl (e.g., 2 to 8 membered, 2 to 6 membered, or 2 to 4 membered), unsubstituted cycloalkyl (e.g., C₃-C₈, C₃-C₆, or C₅-C₆), unsubstituted heterocycloalkyl (e.g., 3 to 8 membered, 3 to 6 membered, or 5 to 6 membered), unsubstituted aryl (e.g., C₆-C₁₀ or phenyl), unsubstituted heteroaryl (e.g., 5 to 10 membered, 5 to 9 membered, or 5 to 6 membered).

R⁴, R⁵, R⁶, R^{4A}, R^{5A}, R^{6A}, R^{4B}, R^{5B} or R^{6B} may be independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

R⁴, R⁵, R⁶, R^{4A}, R^{5A}, R^{6A}, R^{4B}, R^{5B} or R^{6B} may be independently substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴, R⁵, R⁶, R^{4A}, R^{5A}, R^{6A}, R^{4B}, R^{5B} or R^{6B} are independently hydrogen, unsubstituted alkyl, unsubstituted heteroalkyl, unsubstituted cycloalkyl, unsubstituted heterocycloalkyl, unsubstituted aryl, or unsubstituted heteroaryl.

In embodiments R¹ is R^{1A}-substituted C₁-C₂₅ alkyl. Where R¹ is R^{1A}-substituted C₁-C₂₅ alkyl, R^{1A} is independently hydrogen, —N(R^{4A})(R^{5A}), —N(R^{4A})(R^{5A})(R^{6A}), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. And R^{4A}, R^{5A} and R^{6A} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments R^{1A} is hydrogen. In embodiments R^{1A} is —N(R^{4A})(R^{5A}). In embodiments, R^{1A}—N⁺(R^{4A})(R^{5A})(R^{6A}). In embodiments R^{1A} is substituted alkyl. In embodiments R^{1A} is unsubstituted alkyl. In embodiments R^{1A} is substituted heteroalkyl. In embodiments R^{1A} is unsubstituted heteroalkyl. In embodiments R^{1A} is substituted cycloalkyl. In embodiments R^{1A} is unsubstituted cycloalkyl. In embodiments R^{1A} is substituted heterocycloalkyl. In embodiments R^{1A} is unsubstituted heterocycloalkyl. In embodiments R^{1A} is substituted aryl. In embodiments R^{1A} is unsubstituted aryl. In embodiments R^{1A} is substituted heteroaryl. In embodiments R^{1A} is unsubstituted heteroaryl.

In embodiments R^{4A}, R^{5A} and R^{6A} are independently hydrogen. In embodiments R^{4A}, R^{5A} and R^{6A} are independently substituted alkyl. In embodiments R^{4A}, R^{5A} and R^{6A} are independently unsubstituted alkyl. In embodiments R^{4A}, R^{5A} and R^{6A} are independently substituted heteroalkyl. In embodiments R^{4A}, R^{5A} and R^{6A} are independently unsubstituted heteroalkyl. In embodiments R^{4A}, R^{5A} and R^{6A} are independently substituted cycloalkyl. In embodiments R^{4A}, R^{5A} and R^{6A} are independently unsubstituted cycloalkyl. In embodiments R^{4A}, R^{5A} and R^{6A} are independently substituted heterocycloalkyl. In embodiments R^{4A}, R^{5A} and R^{6A} are independently unsubstituted heterocycloalkyl. In embodiments R^{4A}, R^{5A} and R^{6A} are independently substituted aryl. In embodiments R^{4A}, R^{5A} and R^{6A} are independently unsubstituted aryl. In embodiments R^{4A}, R^{5A} and R^{6A} are independently substituted heteroaryl. In embodiments R^{4A}, R^{5A} and R^{6A} are independently unsubstituted heteroaryl.

In embodiments, R^{1A} is unsubstituted C₁-C₂₅ alkyl. In embodiments, R^{1A} is unsubstituted C₂-C₂₅ alkyl. In embodiments, R^{1A} is unsubstituted C₃-C₂₅ alkyl. In embodiments, R^{1A} is unsubstituted C₄-C₂₅ alkyl. In embodiments, R^{1A} is unsubstituted C₅-C₂₅ alkyl. In embodiments, R^{1A} is unsubstituted C₆-C₂₅ alkyl. In embodiments, R^{1A} is unsubstituted C₇-C₂₅ alkyl. In embodiments, R^{1A} is unsubstituted C₈-C₂₅ alkyl. In embodiments, R^{1A} is unsubstituted C₉-C₂₅ alkyl. In embodiments, R^{1A} is unsubstituted C₁₀-C₂₅ alkyl. In embodiments, R^{1A} is unsubstituted C₁₁-C₂₅ alkyl. In embodiments, R^{1A} is unsubstituted C₁₀-C₂₅ alkyl. In embodiments, R^{1A} is unsubstituted C₁₃-C₂₅ alkyl. In embodiments, R^{1A} is unsubstituted C₁₄-C₂₅ alkyl. In embodiments, R^{1A} is unsubstituted C₁₅-C₂₅ alkyl. In embodiments, R^{1A} is unsubstituted C₁₆-C₂₅ alkyl. In embodiments, R^{1A} is unsubstituted C₁₇-C₂₅ alkyl. In embodiments, R^{1A} is unsubstituted C₁₈-C₂₅ alkyl. In embodiments, R^{1A} is unsubstituted C₁₉-C₂₅ alkyl. In embodiments, R^{1A} is unsubstituted C₂₀-C₂₅ alkyl. In embodiments, R^{1A} is unsubstituted C₂₁-C₂₅ alkyl. In embodiments, R^{1A} is unsubstituted C₂₂-C₂₅ alkyl. In embodiments, R^{1A} is unsubstituted C₂₃-C₂₅ alkyl.

In embodiments, R^{1A} is unsubstituted C₁-C₂₄ alkyl. In embodiments, R^{1A} is unsubstituted C₁-C₂₃ alkyl. In embodiments, R^{1A} is unsubstituted C₁-C₂₂ alkyl. In embodiments, R^{1A} is unsubstituted C₁-C₂₁ alkyl. In embodiments, R^{1A} is unsubstituted C₁-C₂₀ alkyl. In embodiments, R^{1A} is unsubstituted C₁-C₁₉ alkyl. In embodiments, R^{1A} is unsubstituted C₁-C₁₈ alkyl. In embodiments, R^{1A} is unsubstituted C₁-C₁₇ alkyl. In embodiments, R^{1A} is unsubstituted C₁-C₁₆ alkyl. In embodiments, R^{1A} is unsubstituted C₁-C₁₅ alkyl. In embodiments, R^{1A} is unsubstituted C₁-C₁₄ alkyl. In embodiments, R^{1A} is unsubstituted C₁-C₁₃ alkyl. In embodiments, R^{1A} is unsubstituted C₁-C₁₂ alkyl. In embodiments, R^{1A} is unsubstituted C₁-C₁₁ alkyl. In embodiments, R^{1A} is unsubstituted C₁-C₁₀ alkyl. In embodiments, R^{1A} is unsubstituted C₁-C₉ alkyl. In embodiments, R^{1A} is unsubstituted C₁-C₈ alkyl. In embodiments, R^{1A} is unsubstituted C₁-C₇ alkyl. In embodiments, R^{1A} is unsubstituted C₁-C₆ alkyl. In embodiments, R^{1A} is unsubstituted C₁-C₅ alkyl. In embodiments, R^{1A} is unsubstituted C₁-C₄ alkyl. In embodiments, R^{1A} is unsubstituted C₁-C₃ alkyl. In embodiments, R^{1A} is unsubstituted C₂₅ alkyl, C₂₄ alkyl, C₂₃ alkyl, C₂₂ alkyl, C₂₁ alkyl, C₂₀ alkyl, C₁₉ alkyl, C₁₈ alkyl, C₁₇ alkyl, C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₆ alkyl, C₅ alkyl, C₄ alkyl, C₃ alkyl, C₂ alkyl or C₁ alkyl.

In embodiments R^{1A} is —N(R^{4A})(R^{5A}) or —N(R^{4A})(R^{5A})(R^{6A}) and R^{4A}, R^{5A} and R^{6A} are independently substituted or unsubstituted C₁-C₅ alkyl. In embodiments R^{1A} is —N(R^{4A})(R^{5A}). In embodiments R^{1A} is —N⁺(R^{4A})(R^{5A})(R^{6A}). In embodiments, R^{4A}, R^{5A} and R^{6A} are independently unsubstituted C₁-C₅ alkyl. In embodiments, R^{4A}, R^{5A} and R^{6A} are independently unsubstituted C₂-C₅ alkyl. In embodiments, R^{4A}, R^{5A} and R^{6A} are independently unsubstituted C₃-C₅ alkyl.

In embodiments, R^{4A}, R^{5A} and R^{6A} are independently unsubstituted C₁-C₄ alkyl. In embodiments, R^{4A}, R^{5A} and R^{6A} are independently unsubstituted C₁-C₃ alkyl. In embodiments, R^{4A}, R^{5A} and R^{6A} are independently unsubstituted C₅ alkyl, C₄ alkyl, C₃ alkyl, C₂ alkyl or C₁ alkyl.

In embodiments, R^{4A}, R^{5A} and R^{6A} are independently substituted C₁-C₅ alkyl. In embodiments, R^{4A}, R^{5A} and R^{6A} are independently substituted C₂-C₅ alkyl. In embodiments, R^{4A}, R^{5A} and R^{6A} are independently substituted C₃-C₅ alkyl.

In embodiments, R^{4A}, R^{5A} and R^{6A} are independently substituted C₁-C₄ alkyl. In embodiments, R^{4A}, R^{5A} and R^{6A} are independently substituted C₁-C₃ alkyl. In embodiments, R^{4A}, R^{5A} and R^{6A} are independently substituted C₅ alkyl, C₄ alkyl, C₃ alkyl, C₂ alkyl or C₁ alkyl. In embodiments, R^{4A}, R^{5A} and R^{6A} are independently unsubstituted C₁ alkyl.

In embodiments, R^{1A} is unsubstituted 5-10-membered aryl. In embodiments, R^{1A} is unsubstituted 5-membered aryl. In embodiments, R^{1A} is unsubstituted 6-membered aryl. In embodiments, R^{1A} is unsubstituted 7-membered aryl. In embodiments, R^{1A} is unsubstituted 8-membered aryl. In embodiments, R^{1A} is unsubstituted 9-membered aryl. In embodiments, R^{1A} is unsubstituted 10-membered aryl.

In embodiments, R^{1A} is unsubstituted phenyl or unsubstituted naphthyl. In embodiments, R^{1A} is unsubstituted phenyl. In embodiments, R^{1A} is unsubstituted naphthyl.

In embodiments R¹ is substituted or unsubstituted 5-10-membered aryl. In embodiments R¹ is substituted 5-membered aryl. In embodiments R¹ is substituted 6-membered aryl. In embodiments R¹ is substituted 7-membered aryl. In embodiments R¹ is substituted 8-membered aryl. In embodiments R¹ is substituted 9-membered aryl. In embodiments R¹ is substituted 10-membered aryl.

In embodiments R¹ is unsubstituted 5-membered aryl. In embodiments R¹ is unsubstituted 6-membered aryl. In embodiments R¹ is unsubstituted 7-membered aryl. In embodiments R¹ is unsubstituted 8-membered aryl. In embodiments R¹ is unsubstituted 9-membered aryl. In embodiments R¹ is unsubstituted 10-membered aryl.

In embodiments, R¹ is substituted or unsubstituted phenyl. In embodiments, R¹ is R^{1A}-substituted phenyl. In embodiments, R¹ is unsubstituted phenyl.

In embodiments, R¹ is substituted or unsubstituted naphthyl. In embodiments, R¹ is R^{1A}-substituted naphthyl. In embodiments R¹ is unsubstituted naphthyl.

In embodiments, R¹ is R^{1A}-substituted 5-10-membered aryl, wherein R^{1A} is unsubstituted C₁-C₂₅ alkyl. Per the above, R^{1A} may be for example, unsubstituted C₁, C₂, C₃, C₄, C₅, C₆, C₇, C₈, C₉, C₁₀, C₁₁, CU, C₁₃, C₁₄, C₁₅, C₁₆, C₁₇, C₁₈, C₁₉, C₂₀, C₂₁, C₂₂, C₂₃, C₂₄, or C₂₅ alkyl. In embodiments, R^{1A}-substituted is 5-membered aryl. In embodiments, R^{1A}-substituted is 6-membered aryl. In embodiments, R^{1A}-substituted is 7-membered aryl. In embodiments, R^{1A}-substituted is 8-membered aryl. In embodiments, R^{1A}-substituted is 9-membered aryl. In embodiments, R^{1A}-substituted is 10-membered aryl. In embodiments, R¹ is R^{1A}-substituted phenyl and R^{1A} is unsubstituted C₈ alkyl.

In embodiments, R² is —SR³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is —SR³. In embodiments, R² is substituted alkyl. In embodiments, R² is unsubstituted alkyl. In embodiments, R² is substituted heteroalkyl. In embodiments, R² is unsubstituted heteroalkyl. In embodiments, R² is substituted cycloalkyl. In embodiments, R² is unsubstituted cycloalkyl. In embodiments, R² is substituted heterocycloalkyl. In embodiments, R² is unsubstituted heterocycloalkyl. In embodiments, R² is substituted aryl. In embodiments, R² is unsubstituted aryl. In embodiments, R² is substituted heteroaryl. In embodiments, R² is unsubstituted heteroaryl.

In embodiments, R² is hydrogen, substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R² is hydrogen, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is substituted alkyl. In embodiments, R³ is unsubstituted alkyl. In embodiments, R³ is substituted heteroalkyl. In embodiments, R³ is unsubstituted heteroalkyl. In embodiments, R³ is substituted cycloalkyl. In embodiments, R³ is unsubstituted cycloalkyl. In embodiments, R³ is substituted heterocycloalkyl. In embodiments, R³ is unsubstituted heterocycloalkyl. In embodiments, R³ is substituted aryl. In embodiments, R³ is unsubstituted aryl. In embodiments, R³ is substituted heteroaryl. In embodiments, R³ is unsubstituted heteroaryl.

In embodiments, R² is —SR³ or substituted or unsubstituted heteroalkyl.

In embodiments, R² is substituted 2-8 membered heteroalkyl. In embodiments, R² is substituted 2 membered heteroalkyl. In embodiments, R² is substituted 3 membered heteroalkyl. In embodiments, R² is substituted 4 membered heteroalkyl. In embodiments, R² is substituted 5 membered heteroalkyl. In embodiments, R² is substituted 6 membered heteroalkyl. In embodiments, R² is substituted 7 membered heteroalkyl. In embodiments, R² is substituted 8 membered heteroalkyl.

In embodiments, R² is substituted 4 membered heteroalkyl.

In embodiments, R² is

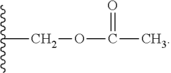 [SMILES: *COC(C)=O]

In embodiments, R² is —SR³ and R³ is substituted or unsubstituted C₁-C₅ alkyl. In embodiments, R³ is unsubstituted C₁-C₅ alkyl. In embodiments, R³ is unsubstituted C₂-C₅ In embodiments, R³ is unsubstituted C₃-C₅ alkyl.

In embodiments, R³ is hydrogen, substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R³ is hydrogen, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is unsubstituted C₁-C₁₂ alkyl. In embodiments, R³ is unsubstituted C₁-C₁₁ alkyl. In embodiments, R³ is unsubstituted C₁-C₁₀ alkyl. In embodiments, R³ is unsubstituted C₁-C₉ alkyl. In embodiments, R³ is unsubstituted C₁-C₈ alkyl. In embodiments, R³ is unsubstituted C₁-C₇ alkyl. In embodiments, R³ is unsubstituted C₁-C₆ alkyl. In embodiments, R³ is unsubstituted C₁-C₅ alkyl. In embodiments, R³ is unsubstituted C₁-C₄ alkyl.

In embodiments, R³ is unsubstituted C₄-C₁₂ alkyl. In embodiments, R³ is unsubstituted C₃-C₁₂ alkyl. In embodiments, R³ is unsubstituted C₁-C₁₂ alkyl. In embodiments, R³ is unsubstituted C₅-C₁₂ alkyl. In embodiments, R³ is unsubstituted C₆-C₁₂ alkyl. In embodiments, R³ is unsubstituted C₇-C₁₂ alkyl. In embodiments, R³ is unsubstituted C₈-C₁₂ alkyl. In embodiments, R³ is unsubstituted C₉-C₁₂ alkyl. In embodiments, R³ is unsubstituted C₁₀-C₁₂ alkyl. In embodiments, R³ is unsubstituted C₁, C₂, C₃, C₄, C₅, C₆, C₇, C₈, C₈, C₉, C₁₀, C₁₁ or C₁₂ alkyl.

In embodiments R² is —SR³ and R³ is substituted or unsubstituted C₅-C₁₀ aryl. In embodiments R³ is substituted C₅ aryl. In embodiments R³ is substituted C₆ aryl. In embodiments R³ is substituted C₇ aryl. In embodiments R³ is substituted C₈ aryl. In embodiments R³ is substituted C₉ aryl. In embodiments R³ is substituted C₁₀. In embodiments R³ is unsubstituted C₅ aryl. In embodiments R³ is unsubstituted C₆ aryl. In embodiments R³ is unsubstituted C₇ aryl. In embodiments R³ is unsubstituted C₈ aryl. In embodiments R³ is unsubstituted C₉ aryl. In embodiments R³ is substituted or unsubstituted C₁₀.

In embodiments R³ is unsubstituted phenyl.

In embodiments R² is —SR³ and R³ is substituted or unsubstituted 5 to 10 membered heteroaryl. In embodiments R³ is substituted 5 membered heteroaryl. In embodiments R³ is substituted 6 membered heteroaryl. In embodiments R³ is substituted 7 membered heteroaryl. In embodiments R³ is substituted 8 membered heteroaryl. In embodiments R³ is substituted 9 membered heteroaryl. In embodiments R³ is substituted 10 membered heteroaryl. In embodiments R³ is unsubstituted 5 membered heteroaryl. In embodiments R³ is unsubstituted 6 membered heteroaryl. In embodiments R³ is unsubstituted 7 membered heteroaryl. In embodiments R³ is unsubstituted 8 membered heteroaryl. In embodiments R³ is unsubstituted 9 membered heteroaryl. In embodiments R³ is unsubstituted 10 membered heteroaryl.

In embodiments R³ is unsubstituted pyridyl.

In embodiments L¹ is a bond, substituted or unsubstituted alkylene or

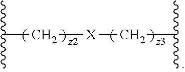

In embodiments L¹ is a bond. In embodiments L¹ is substituted or unsubstituted alkylene. In embodiments L¹ is

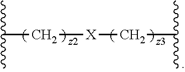

And X is a bond, —S—, —O—, —NH—, —C(O)—NH— or —C(O)—.
And z2 and z3 are independently integers from 0 to 25.

In embodiments X is —C(O)—NH—.

In embodiments z1 is 1 or 2. In embodiments z1 is 1. In embodiments z1 is 2. In embodiments z1 is 0. In embodiments z1 is 3. In embodiments z1 is 4. In embodiments z1 is 5.

In embodiments z2 is 0 or 1. In embodiments z2 is 0. In embodiments z2 is 1.

In embodiments z3 is 0, 1, 2 or 4. In embodiments z3 is 0. In embodiments z3 is 1. In embodiments z3 is 2. In embodiments z3 is 3. In embodiments z3 is 4.

In embodiments z3 is an integer from 10-15. In embodiments z3 is 10. In embodiments z3 is 11. In embodiments z3 is 12. In embodiments z3 is 13. In embodiments z3 is 14. In embodiments z3 is 15. In embodiments z3 is 1, 2, 3, 4, 5, 6, 7, 8, 9, 10, 11, 12, 13, 14 or 15.

In embodiments, z2 and z3 are independently integers from 0 to 25. In embodiments, z2 and z3 are independently integers from 1 to 25. In embodiments, z2 and z3 are independently integers from 2 to 25. In embodiments, z2 and z3 are independently integers from 3 to 25. In embodiments, z2 and z3 are independently integers from 4 to 25. In embodiments, z2 and z3 are independently integers from 5 to 25. In embodiments, z2 and z3 are independently integers from 6 to 25. In embodiments, z2 and z3 are independently integers from 7 to 25. In embodiments, z2 and z3 are independently integers from 8 to 25. In embodiments, z2 and z3 are independently integers from 9 to 25. In embodiments, z2 and z3 are independently integers from 10 to 25. In embodiments, z2 and z3 are independently integers from 11 to 25. In embodiments, z2 and z3 are independently integers from 12 to 25. In embodiments, z2 and z3 are independently integers from 13 to 25. In embodiments, z2 and z3 are independently integers from 14 to 25. In embodiments, z2 and z3 are independently integers from 15 to 25. In embodiments, z2 and z3 are independently integers from 16 to 25. In embodiments, z2 and z3 are independently integers from 17 to 25. In embodiments, z2 and z3 are independently integers from 18 to 25. In embodiments, z2 and z3 are independently integers from 19 to 25. In embodiments, z2 and z3 are independently integers from 20 to 25. In embodiments, z2 and z3 are independently integers from 21 to 25. In embodiments, z2 and z3 are independently integers from 22 to 25. In embodiments, z2 and z3 are independently integers from 23 to 25.

In embodiments, z2 and z3 are independently integers from 1, 2, 3, 4, 5, 6, 7, 8, 9, 10, 11, 12, 13, 14, 15, 16 17, 18, 19, 20, 21, 22, 23, 24 or 25.

In embodiments, L¹ is a bond, —S(O)₂—, —NH—, —O—, —S—, —C(O)—, —C(O)NH—, C(O)CH₂—, —NHC(O)—, —NHC(O)NH—, —C(O)O—, —OC(O)—, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene. In embodiments, L¹ is a bond, substituted (e.g., substituted with a substituent group, a size-limited substituent group, or lower substituent group) or unsubstituted alkylene, substituted (e.g., substituted with a substituent group, a size-limited substituent group, or lower substituent group) or unsubstituted heteroalkylene, substituted (e.g., substituted with a substituent group, a size-limited substituent group, or lower substituent group) or unsubstituted cycloalkylene, substituted (e.g., substituted with a substituent group, a size-limited substituent group, or lower substituent group) or unsubstituted heterocycloalkylene, substituted (e.g., substituted with a substituent group, a size-limited substituent group, or lower substituent group) or unsubstituted arylene, or substituted (e.g., substituted with a substituent group, a size-limited substituent group, or lower substituent group) or unsubstituted heteroarylene.

In embodiments, L¹ is a bond, —S(O)₂—, —NH—, —O—, —S—, —C(O)—, —C(O)NH—, —C(O)CH₂—, —NHC(O)—, —NHC(O)NH—, —C(O)O—, —OC(O)—, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered). In embodiments, L¹ is a bond, substituted or unsubstituted alkylene (e.g., C₁-C₈, C₁-C₆, or C₁-C₄), substituted or unsubstituted heteroalkylene (e.g., 2 to 8 membered, 2 to 6 membered, or 2 to 4 membered), substituted or unsubstituted cycloalkylene (e.g., C₃-C₈, C₃-C₆, or C₅-C₆), substituted or unsubstituted heterocycloalkylene (e.g., 3 to 8 membered, 3 to 6 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

In embodiments, L¹ is a bond or unsubstituted C₁-C₈ alkylene. In embodiments L¹ is unsubstituted C₂-C₈ alkylene. In embodiments L¹ is unsubstituted C₃-C₈ alkylene. In embodiments L¹ is unsubstituted C₄-C₈ alkylene. In embodiments L¹ is unsubstituted C₅-C₈ alkylene. In embodiments L¹ is unsubstituted C₆-C₈ alkylene. In embodiments L¹ is unsubstituted C₁-C₇ alkylene. In embodiments L¹ is unsubstituted C₆ alkylene. In embodiments L¹ is unsubstituted C₁-C₅ alkylene. In embodiments L¹ is unsubstituted C₄ alkylene. In embodiments L¹ is unsubstituted C₃ alkylene. In embodiments L¹ is C₈, C₇, C₆, C₅, C₄, C₃, C₂ or C₁ alkylene.

In embodiments L¹ is unsubstituted C₂ alkylene or unsubstituted C₄ alkylene. In embodiments L¹ is unsubstituted C₂ alkylene. In embodiments L¹ is unsubstituted C₄ alkylene.

In embodiments, the compound is:

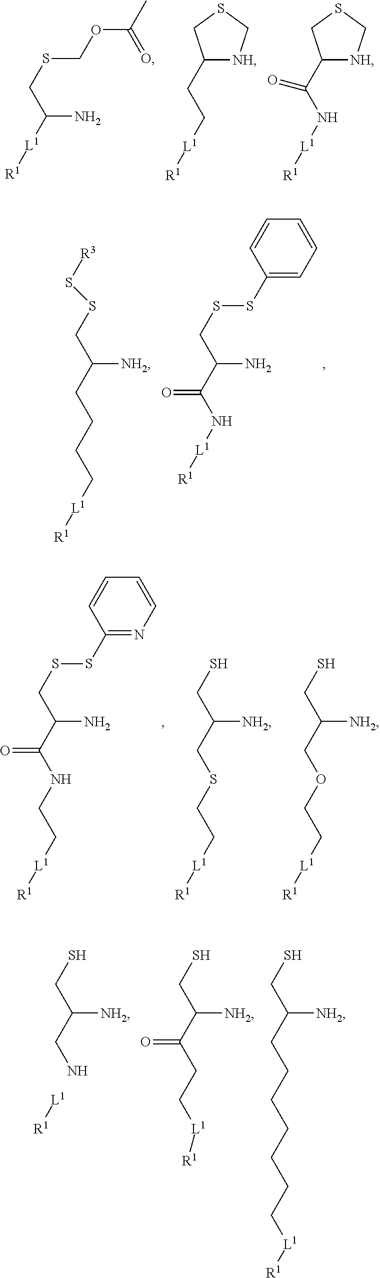

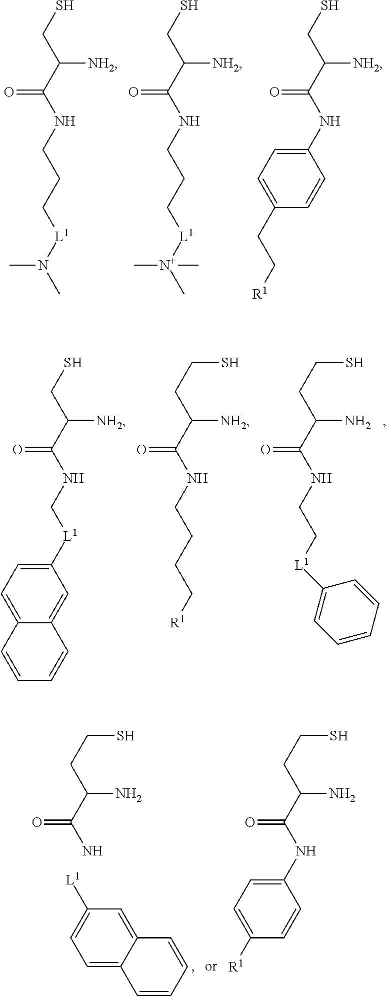

and R¹, R³ and L¹ are defined as herein.

### Pharmaceutical Compositions

In one aspect pharmaceutical compositions including a pharmaceutically acceptable excipient and a compound described herein is provided. For example, the pharmaceutical composition may include any compound of formula (I), (II), or (III).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

In embodiments, the compound is:

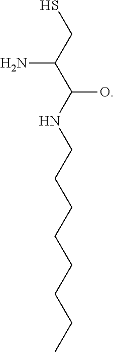

### Methods of Use

The methods provided herein may use any of the compounds provided herein (e.g., any compound of formula (I), (II), or (III)). In one aspect a method of treating a depalmitoylation-associated disease in a subject in need thereof is provided, the method including administering to the subject a therapeutically effective amount of a compound, thereby treating a depalmitoylation-associated disease in the subject.

In embodiments, the depalmitoylation-associated disease is cancer or a neurological disease. In embodiments the depalmitoylation-associated disease is cancer. In embodiments the depalmitoylation-associated disease is a neurological disease.

In embodiments, the depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis or glioma. In embodiments depalmitoylation-associated disease is bladder cancer. In embodiments depalmitoylation-associated disease is head and neck cancer. In embodiments depalmitoylation-associated disease is Costello's Syndrome. In embodiments depalmitoylation-associated disease is melanoma. In embodiments depalmitoylation-associated disease is acute myeloid lymphoma (AML). In embodiments depalmitoylation-associated disease is non-small cell lung carcinoma. In embodiments depalmitoylation-associated disease is Alzheimer's disease. In embodiments depalmitoylation-associated disease is infantile neuronal ceroid lipofuscinosis. In embodiments depalmitoylation-associated disease is glioma.

In another aspect, a method of depalmitoylating a protein in a cell is provided. The method includes contacting the cell with an effective amount of a compound described herein including embodiments thereof. In embodiments the protein forms part of the plasma membrane of the cell. In embodiments, the protein is HRas, NRas, EGFR, amyloid precursor protein (APP), BACE1, EZH2, PD-L1, flotillin-1, flotillin-2, calnexin, Gα(i), metadherin, CD44 or SNAP25. In embodiments the protein is HRas. In embodiments the protein is NRas. In embodiments the protein is EGFR. In embodiments the protein is amyloid precursor protein (APP). In embodiments the protein is BACE1. In embodiments the protein is EZH2. In embodiments the protein is PD-L1. In embodiments the protein is flotillin-1. In embodiments the protein is flotillin-2. In embodiments the protein is calnexin. In embodiments the protein is Gα(i). In embodiments the protein is metadherin. In embodiments the protein is CD44. In embodiments the protein is SNAP25.

In embodiments the contacting occurs in vitro or in vivo. In embodiments the cell forms part of an organism. In embodiments the cell forms part of a mammalian subject. In embodiments the mammalian subject suffers from cancer or a neurological disease.

In an aspect, a method of treating a depalmitoylation-associated disease in a subject in need thereof is provided. The method includes administering to the subject a therapeutically effective amount of a depalmitoylating amphiphilic thiol compound, thereby treating a depalmitoylation-associated disease in the subject. A “depalmitoylating amphiphilic thiol compound” is a compound including an amphiphilic moiety that enables the compound to associate with cellular membranes and a thiol moiety that enables the compound to engage in a native chemical ligation reaction with a palmitoylated protein forming part of said cellular membrane. In embodiments the depalmitoylating amphiphilic thiol compound is a compound described herein.

### EXAMPLES

### Example 1: A Reactive-Amphilphile-Based Strategy for Mimicking Palmitoyl-Protein Thioesterase Activity in Living Cells

Post-translational S-palmitoylation plays a central role in protein localization, trafficking, stability, aggregation, and cell signaling. Dysregulation of palmitoylation pathways in cells can alter protein function and is the cause of several diseases. Considering the biological and clinical importance of S-palmitoylation, tools for direct, in vivo modulation of this lipid modification would be extremely valuable. Here, we describe a method for the cleavage of native S-palmitoyl groups from proteins in living cells. Using a cell permeable, cysteine-functionalized amphiphile, we demonstrate the direct depalmitoylation of cellular proteins. We show that amphiphile-mediated depalmitoylation (AMD) can effectively cleave S-palmitoyl groups from the native GTPase HRas and successfully depalmitoylate mislocalized proteins in an infantile neuronal ceroid lipofuscinosis (INCL) disease model. AMD enables direct and facile depalmitoylation of proteins in live cells and has potential therapeutic applications for diseases such as INCL, where native protein thioesterase activity is deficient.

Cellular proteins undergo numerous post-translational modifications (PTMs), including phosphorylation, nitrosylation, glycosylation, ubiquitination, methylation, and lipidation.¹ These modifications play essential roles in the structure, localization, and activity of proteins in cells, and their dysregulation can result in disease.² Protein S-palmitoylation is an essential PTM in which a palmitate group is linked to proteins through a reversible thioester bond with cysteine residues.^{3,4} Defects in palmitoylation can have devastating biological consequences and are implicated in several disorders, including Alzheimer's disease,⁵ diabetes,⁶ and cancer.³ Despite tremendous interest in S-palmitoylation, methods for in vivo remodeling of protein lipidation are limited in scope.^{3,7} While inhibitors, such as 2-bromopalmitate, block the enzymes responsible for S-palmitoylation, they cannot cleave S-palmitoyl groups from currently lipidated proteins and are known to irreversibly inhibit several enzymes involved in lipid biosynthesis.³ Therefore, we sought to engineer a small molecule for direct and chemoselective depalmitoylation of S-palmitoyl groups in vivo.

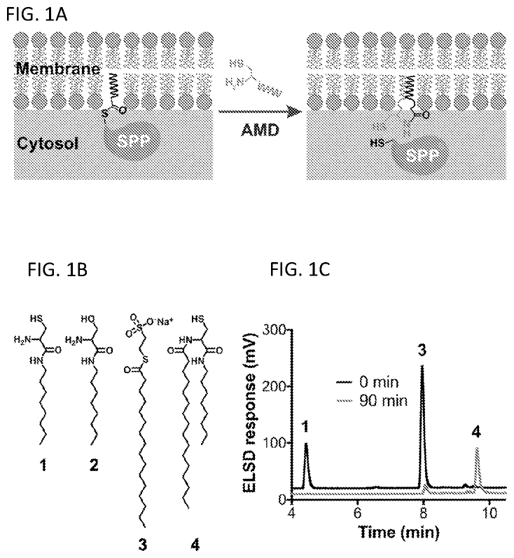

We conceived a method for depalmitoylation of membrane-associated proteins based on the chemoselective reaction of N-terminal cysteines with thioesters through native chemical ligation (NCL)⁸ (FIG. 1A). Previous work in our group has demonstrated that amphiphilic NCL precursors can react spontaneously and rapidly under physiological conditions to yield stable lipid products.⁹⁻¹¹ The small, cysteine-containing amphiphile provided herein may be utilized for the selective depalmitoylation of native membrane-anchored proteins in vivo. The amphiphilic reactant will ensure localization in the membrane, enhancing the reaction rate and selectivity of reaction with membrane-bound proteins bearing thioesters. This amphiphile-mediated depalmitoylation (AMD) approach would offer the advantage of direct cleavage of S-palmitoyl groups from endogenous proteins in a way that is nondestructive towards the rest of the protein.

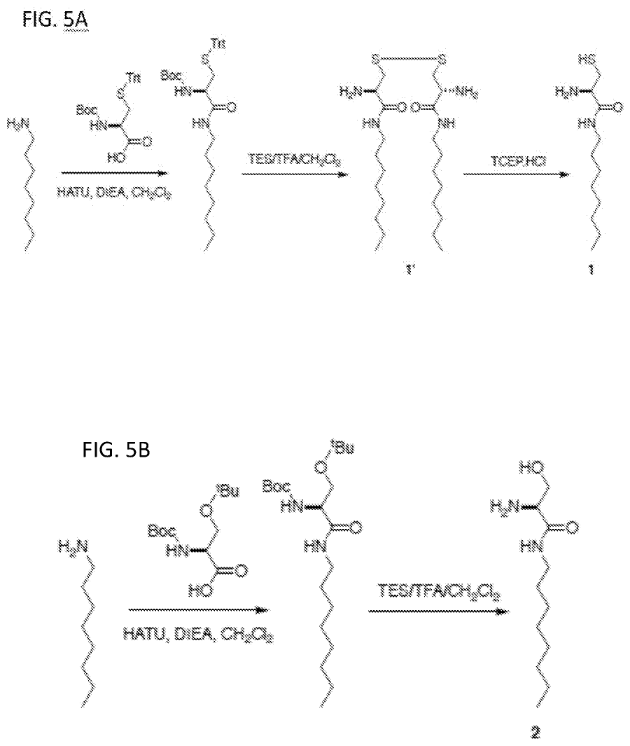

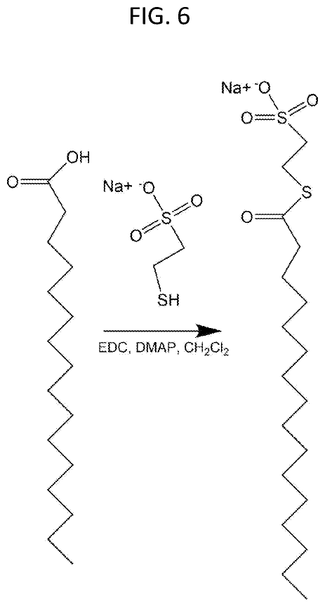

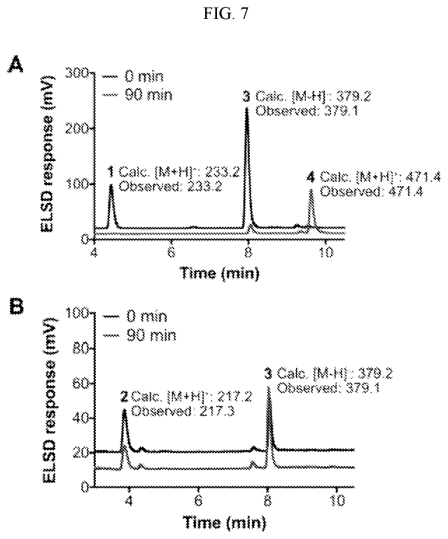

In designing a reactive amphiphile, we sought to balance lipophilicity and aqueous solubility to promote both cell permeability and membrane affinity. Therefore, we employed a lipophilic alkyl chain of moderate length, octylamine, coupled to cysteine for the synthesis of the depalmitoylating agent 1 (FIG. 1B, FIG. 5). To determine if 1 could cleave the thioester linkage of S-palmitoyl groups under physiological conditions, we performed preliminary tests in the presence of S-palmitoyl sodium 2-mercaptoethanesulfonate (MESNA) 3 (FIG. 1B, FIG. 6), an S-palmitoylated protein (SPP) surrogate. We found that the reaction between 1 and 3 proceeded rapidly and irreversibly at ambient temperature and cleaved the model S-palmitoyl group to yield 4 (FIG. 1B), a stable, N-acylated product (FIG. 1C, FIG. 7). To confirm that the observed reaction is due to NCL and not direct aminolysis, we synthesized the alkyl derivative 2, an analog of 1 in which the thiol group is replaced with an alcohol (FIG. 5). Compound 2 should have similar physical properties to 1 but be unable to engage in NCL¹² (FIG. 1B). As expected, the reaction between 2 and 3 under the same conditions yielded no ligation product, demonstrating that the observed reaction proceeds through NCL (FIG. 7).

Having confirmed the efficient reaction of 1 with thioesters in aqueous buffer, we wanted to determine if AMD could be used to cleave palmitoyl groups from S-palmitoylated proteins in vivo. HRas is a member of the Ras family of GTPases, which function as key regulatory proteins in cell differentiation, proliferation, and survival.¹³ Mutations in HRas are associated with several cancers,¹⁴ as well as Costello syndrome, a severe congenital disorder for which there is no cure.^{15,16} Therefore, there is significant interest in developing therapeutics which target HRas signaling.^{17,18} However, Ras proteins are challenging to target and have even been deemed “undruggable”.¹⁷ In mammalian cells, HRas is palmitoylated at the Golgi complex before being trafficked to the plasma membrane. Once at the plasma membrane, HRas can be enzymatically depalmitoylated, triggering its movement back to the Golgi, where it can repeat this cycle.¹⁹ Because of its biological and clinical importance, we chose to target HRas for depalmitoylation by the alkyl cysteine 1.

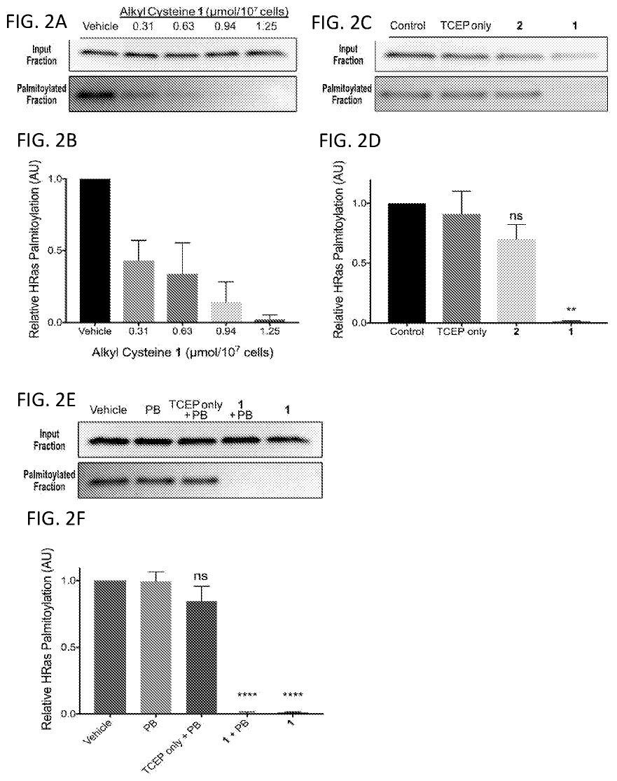

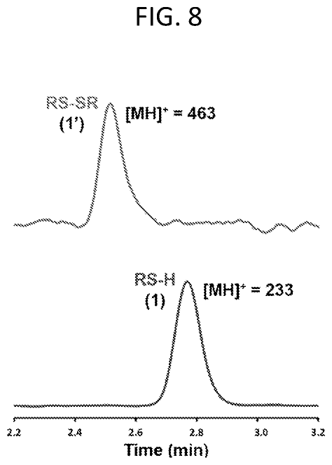

For use in cells, a stock solution of 50 mM 1 or 2 was prepared in DMSO with the addition of 2 equivalents [100 mM] tris(2-carboxyethyl)phosphine hydrochloride (TCEP.HCl), a preservative to prevent oxidation in storage (FIG. 8). Before use, stocks were diluted to the desired concentration in cell media. To determine if 1 can trigger depalmitoylation in live cells, HeLa cells were treated with a range of concentrations of 1 (0.31-1.25 μmol/10⁷ cells) for 20 min. The palmitoylation state of endogenous HRas after treatment with 1 was detected using an acyl resin-assisted capture method (FIG. 2A,B).²⁰ We found that HRas palmitoylation decreased in a dose-dependent manner and near complete depalmitoylation of HRas was observed upon treatment of HeLa cells with 1.25 μmol/10⁷ cells 1. To exclude the possibility that the observed reduction in HRas palmitoylation was due to the preservative TCEP or a non-specific effect of the amphiphile, we performed the same experiments in the presence of TCEP alone or compound 2 (FIG. 2C-D). Under these conditions, no significant depalmitoylation was observed, indicating that the depalmitoylation of HRas is dependent on the cysteine moiety present in 1.

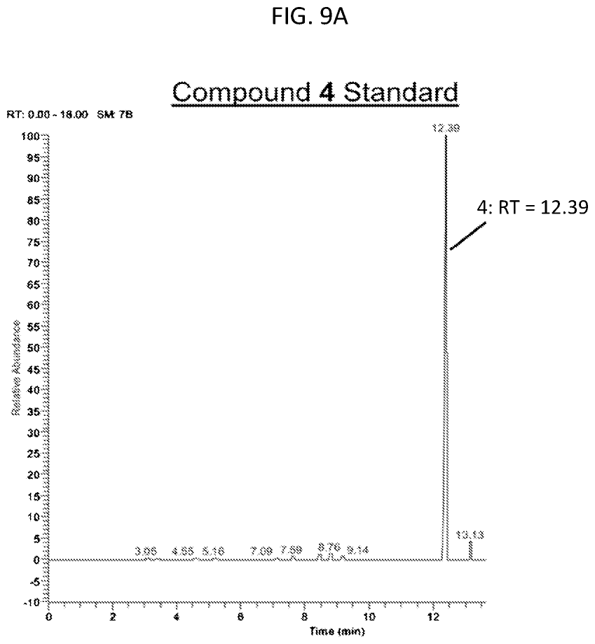

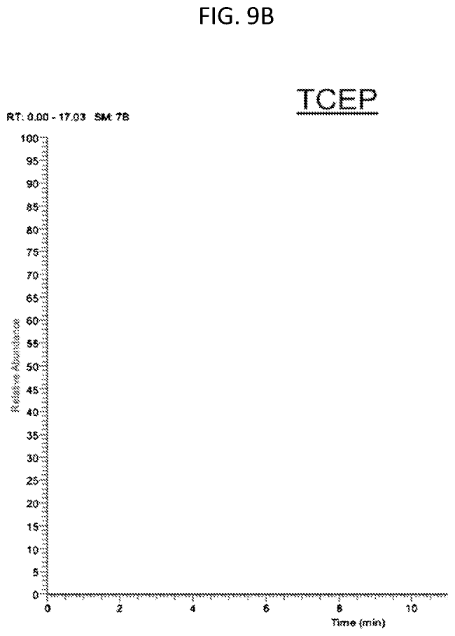

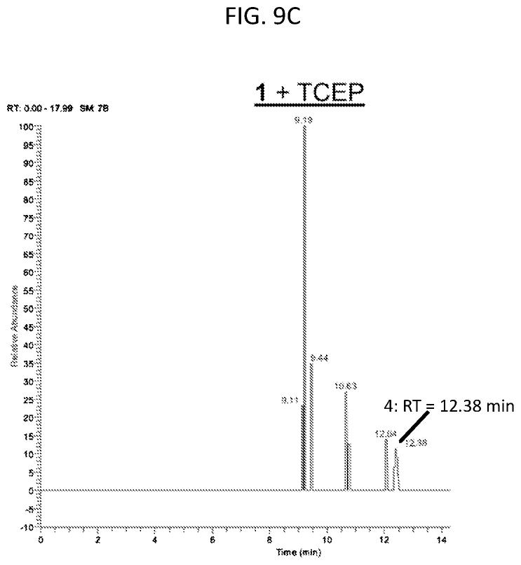

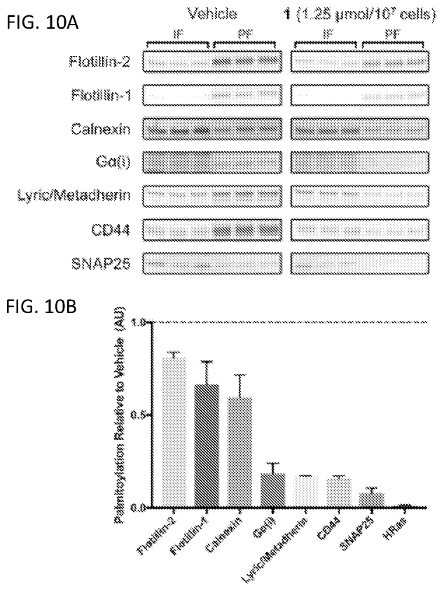

HRas undergoes natural cycles of palmitoylation and depalmitoylation by the palmitoyl acyltransferase DHHC9/GCP16, and acyl protein thioesterases 1 and 2 (APT-1/2), respectively.²¹ To determine if the observed reduction in HRas palmitoylation was due to cleavage of palmitoyl groups by 1, or the result of endogenous thioesterase activity, we treated cells with 1 in the presence of an inhibitor of APT-1/2, palmostatin B (PB).²²′²³ We found that 1 (1.25 μmol/10⁷ cells) efficiently depalmitoylated HRas, even in the presence of PB (FIG. 2E-F), suggesting that it acts by directly cleaving S-palmitoyl groups and not through alternative biological mechanisms. Additionally, no significant depalmitoylation was observed when cells were treated with PB, or PB and TCEP alone (FIG. 2E-F). To confirm that the reaction between 1 and biological thioesters resulted in the expected N-acylated product 4, we treated HeLa cells with 1 and then performed a lipid extraction and mass spectrometric analysis. We detected the expected AMD product, 4, when cells were treated with 1 but not when cells were treated with TCEP preservative alone (FIG. 9). To determine the scope of protein depalmitoylation by AMD in HeLa cells, we detected the palmitoylation levels of eight known palmitoylated proteins after treatment with 1. We found that AMD reduced the palmitoylation level of five of the tested proteins (Gα(i), lyric/metadherin, CD44, SNAP25 and HRas) by >80%. Palmitoylation levels in three other proteins (flotillin-1, flotillin-2 and calnexin), however, were only reduced by 19-40% (FIG. 10). The reduced activity of AMD towards flotillins may be due to their association with membrane rafts, which could restrict access of 1 to their S-palmitoylation site.²⁴ Calnexin, on the other hand, is localized in ER membranes,²⁵ and thus may be less susceptible to depalmitoylation as compared to plasma membrane proteins.

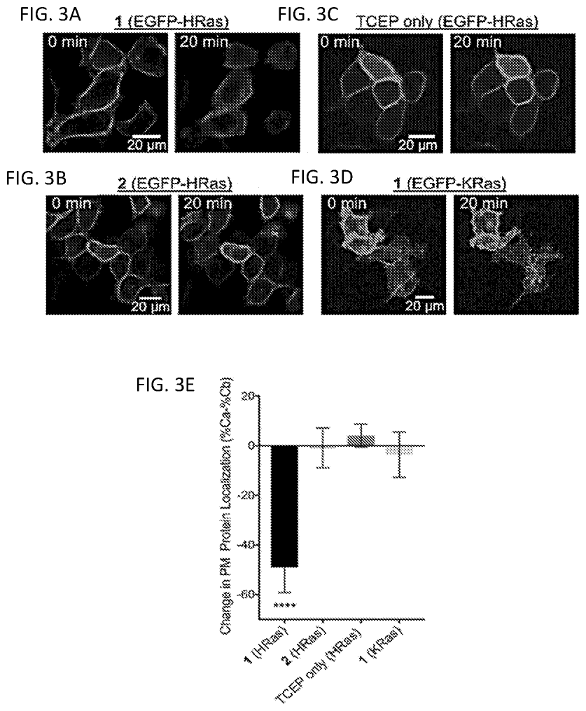

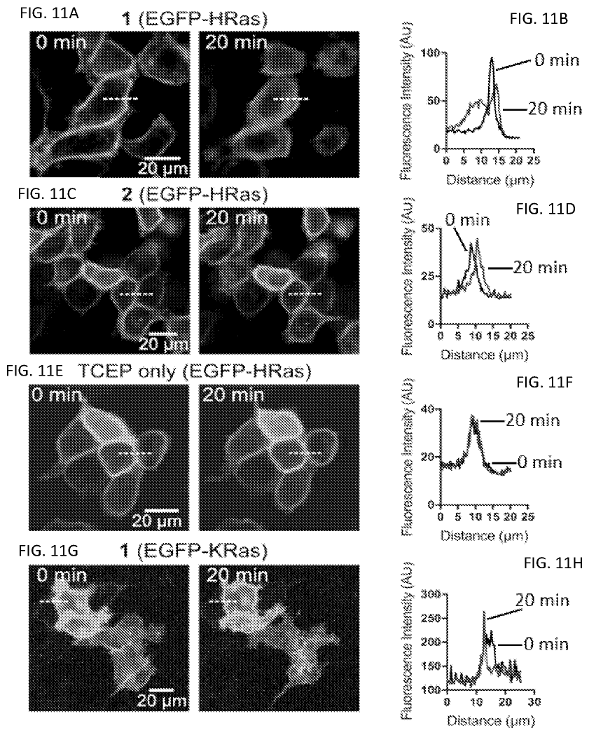

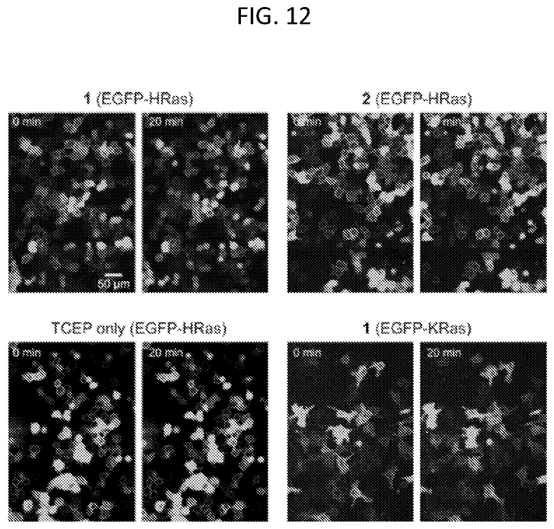

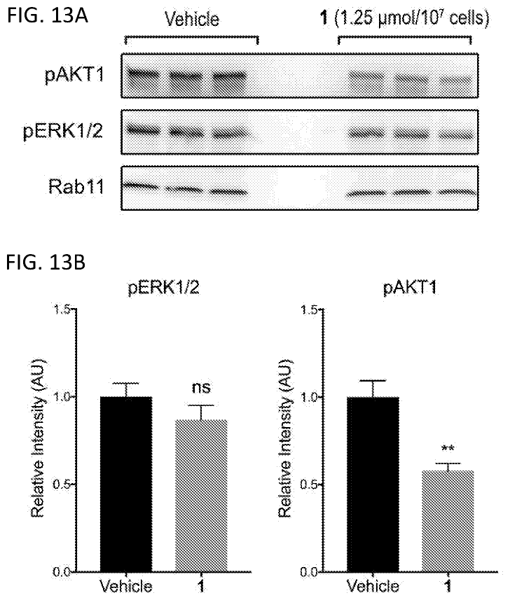

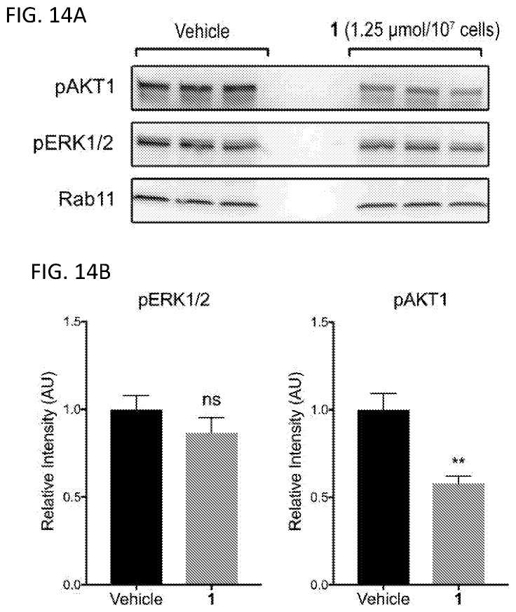

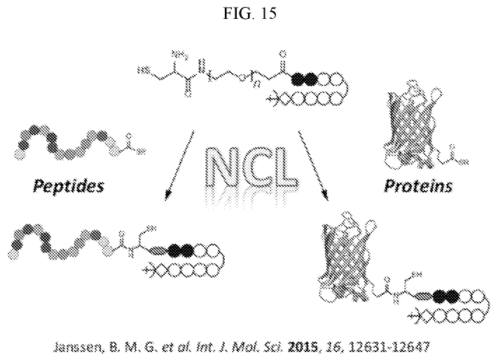

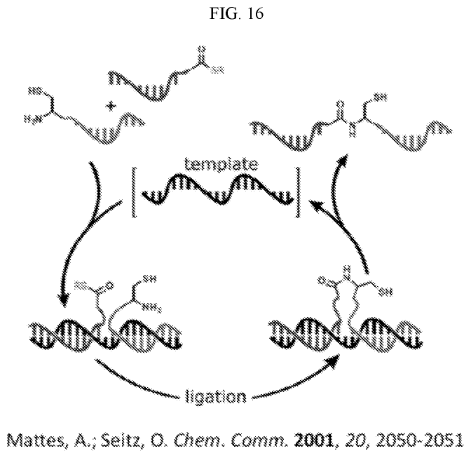

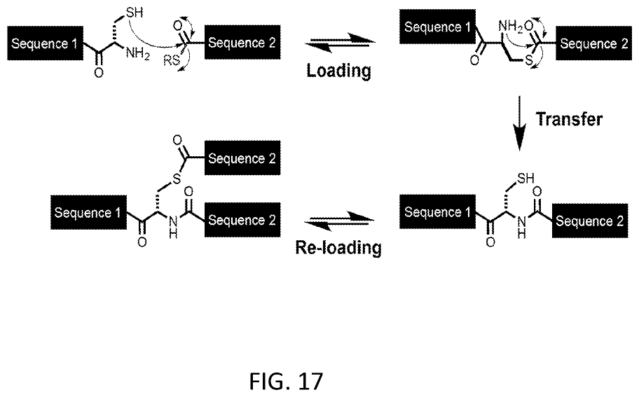

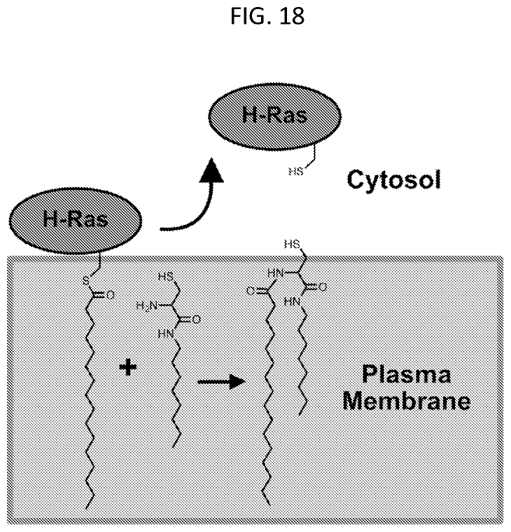

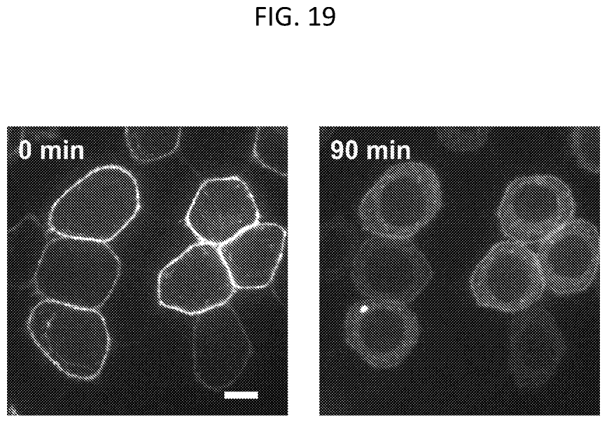

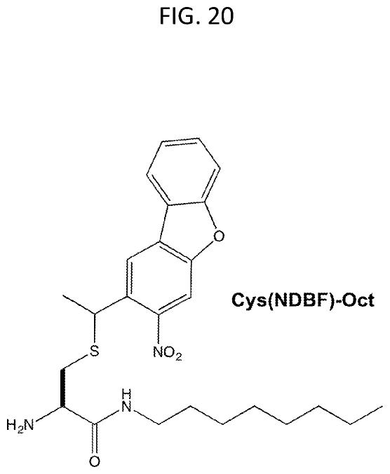

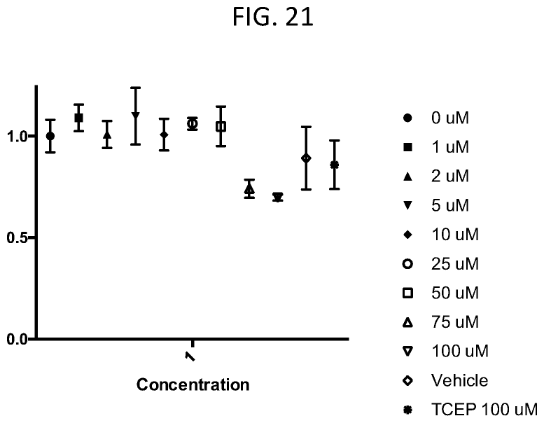

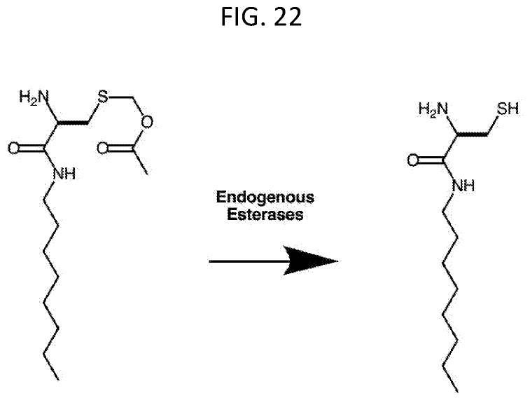

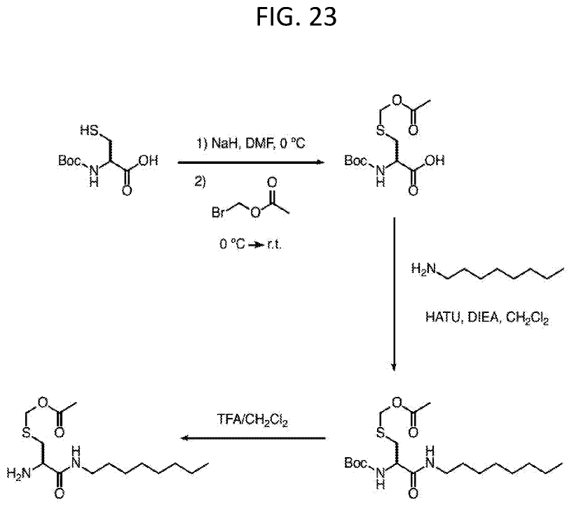

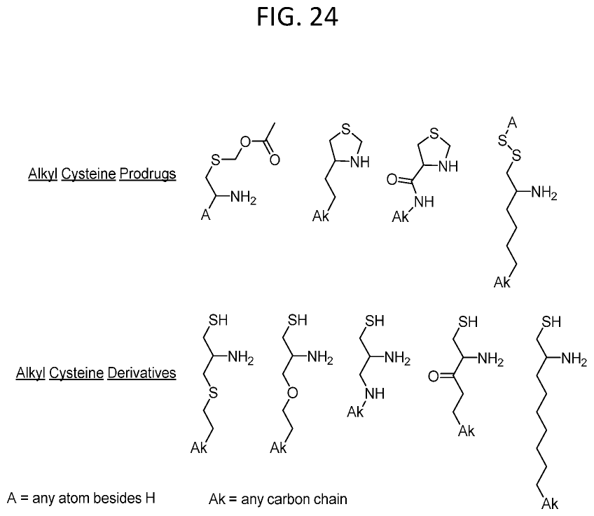

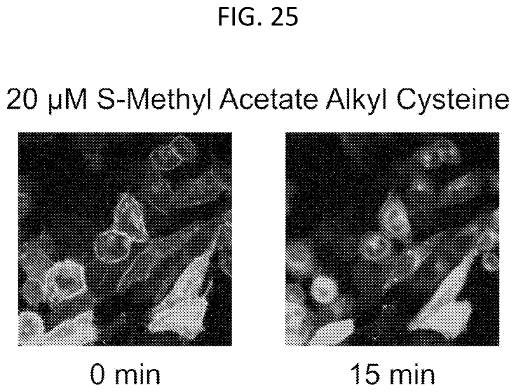

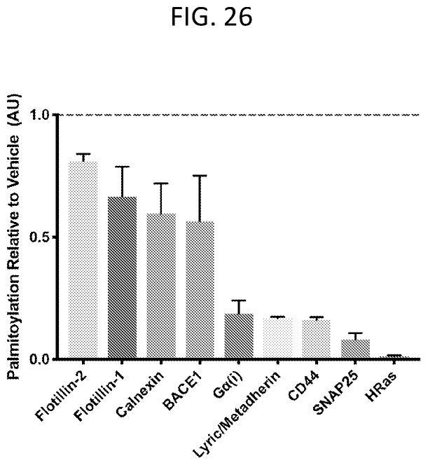

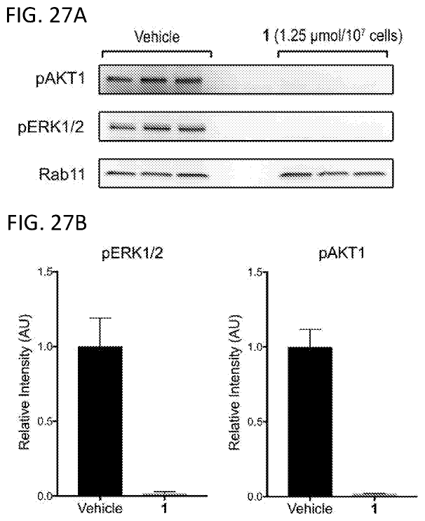

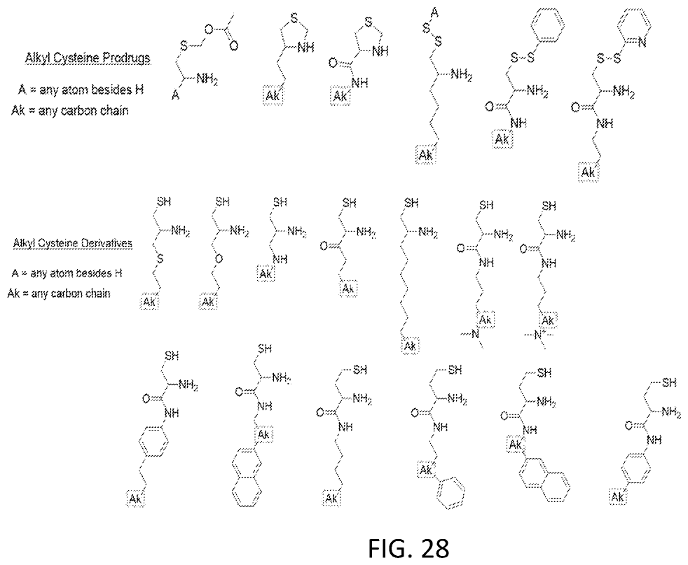

Previous studies have shown that the cysteine palmitoylation sites of HRas are necessary for its localization to the plasma membrane. Mutation of these sites results in a distinct shift in HRas localization from the plasma membrane to ER/Golgi membranes and cytosol.^{19,21} To investigate the ability of AMD to trigger changes in HRas localization, we transiently transfected HeLa cells with a plasmid encoding for EGFP-HRas. Initially, EGFP-HRas was observed primarily at the plasma membrane, indicative of its S-palmitoylated state (FIG. 3A).²¹ After treatment with 1 (1.25 μmol/10⁷ cells) for 20 min, EGFP-HRas localization at the plasma membrane decreased and was accompanied by an increase in fluorescence at inner cellular membranes, adopting a distribution consistent with depalmitoylated HRas (FIG. 3A, FIG. 10).²¹ Controls with compound 2 (FIG. 3B, FIG. 11) or TCEP alone (FIG. 3C, FIG. 11) showed no change in fluorescence localization. Furthermore, cells transfected with plasmid encoding EGFP-KRas4b, a Ras isoform tethered to the plasma membrane by an electrostatic interaction instead of S-palmitoylation, and treated with 1 (1.25 μmol/10⁷ cells), showed no change in protein localization (FIG. 3D, FIG. 11). Statistical analysis of several hundred cells under each condition demonstrated a significant change in plasma membrane protein localization only in EGFP-HRas transfected cells treated with 1 (FIG. 3E, FIG. 12). To determine if treatment with 1 has any effect on Ras signaling pathways, we analyzed the phosphorylation of AKT1 (pS473) and ERK1/2 (pY204/pY187), two downstream effectors regulated by Ras.²⁶ We found that treatment of HeLa cells with 1 (1.25 μmol/10⁷ cells) resulted in a significant decrease in AKT1 phosphorylation, but not ERK1/2 phosphorylation, after EGF stimulation (FIG. 13). Interestingly, treatment of T24 cells, a bladder cancer cell line with an oncogenic HRas mutation,²⁷ with 1 resulted in near complete abrogation of AKT1 and ERK1/2 phosphorylation (FIG. 14). These results suggest that cells with hyperactive HRas may be uniquely susceptible to signaling inhibition by AMD.²⁸

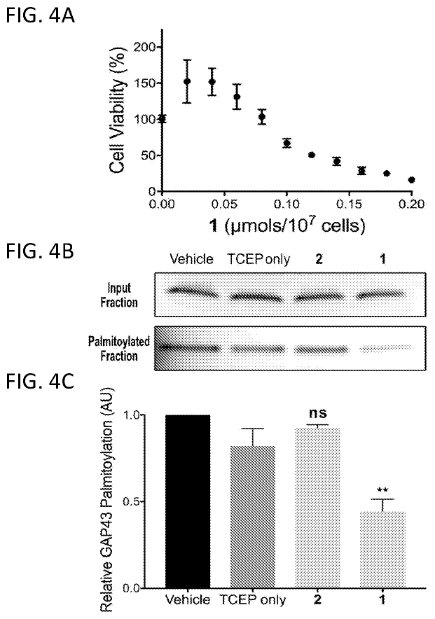

Palmitoylation plays a crucial role in directing protein localization and function, and disruption of these processes can lead to disease.²⁹ Infantile neuronal ceroid lipofuscinosis (INCL) is a degenerative and fatal disease caused by mutations in the palmitoyl-protein thioesterase-1 (PPT1) gene.³⁰ PPT1 is a thioesterase responsible for the depalmitoylation of many S-palmitoylated proteins. Mutations which disrupt its activity result in intracellular accumulation of palmitoylated proteins, leading to cell apoptosis and neurodegeneration.³¹ Although rare, INCL is a devastating disease, and currently there exist few treatment options and no cure.^{32,33} AMD may mimic the thioesterase activity of PPT1 and may help reverse the accumulation of palmitoylated proteins caused by INCL. To evaluate the potential of 1 for the treatment of INCL, we obtained patient-derived INCL lymphoblasts and determined their tolerance of 1 over a 24 h period. We found that doses up to 0.08 μmol/10⁷ cells did not decrease cell viability compared to the nontreated control (FIG. 4A). To determine if AMD could decrease the accumulation of S-palmitoylated proteins in INCL cells,^{30,31} we treated INCL lymphoblasts with 1 (0.05 μmol/10⁷ cells) for 24 h. We then detected the palmitoylation level of GAP43, a protein which accumulates at the ER in INCL cells³¹ and is known to have increased levels of palmitoylation in PPT1 knockout models.³⁰ We found that 1 significantly reduced the level of GAP43 palmitoylation in INCL cells (FIG. 4B,C). Treatment of INCL lymphoblasts with TCEP only or 2 did not result in a significant change in GAP43 palmitoylation. This suggests that AMD may help to counteract the increased protein palmitoylation and ER accumulation associated with INCL and could offer a therapeutic benefit.

In conclusion, we have developed a small-molecule depalmitoylation strategy for the cleavage of S-palmitoyl groups in vivo. The efficacy of AMD in vivo and its ability to hit disease-relevant targets suggest it has potential therapeutic value.

Materials and Methods

HPLC Analysis

HPLC analysis was performed on an Agilent 1260 Infinity system equipped with a Varian 380-LC evaporative light scattering detector (ELSD) and an Agilent 6120 Single Quad MS.

AMD Model Reaction

H₂N-L-Cys-Oct U H₂N-L-Cys-Oct (1′) or H₂N-L-Ser-Oct (2) was combined with MESNA thiopalmitate (3) in NaH₂PO₄ buffer (200 mM, pH 7.1) containing 10 mM tris(2-carboxyethyl)phosphine hydrochloride (TCEP) at a final concentration of 5 mM for each reactant. The reaction was stirred at 25° C. and aliquots taken at the specified time points and subjected to HPLC/ELSD/MS analysis (Eclipse Plus C8 analytical column, 50-95% MeOH in H₂O with 0.1% TFA, 0-7 min; 95% MeOH in H₂O with 0.1% TFA, 7 min-end).

Microscopy

Imaging was performed on an Axio Observer Z1 inverted microscope (Carl Zeiss Microscopy Gmb, Germany) with Yokogawa CSU-X1 spinning disk confocal unit using a 63×, 1.4 NA oil immersion or 20×, 0.8 NA objective to an ORCA-Flash4.0 V2 Digital CMOS camera (Hamamatsu, Japan). For live cell imaging, an incubation chamber with temperature and CO₂ controllers (World Precision Instruments) was utilized. Fluorophores were excited with s diode laser (488 nm; 30 mW). Images were acquired using Zen Blue software (Carl Zeiss) and processed using Image J.¹

Cell Culture

Infantile Neuronal Ceroid Lipofuscinosis (INCL) human lymphoblasts (GM16083) were obtained from Coriell Institute (Camden, N.J.). T24 cells (ATCC HTB-4) were obtained from ATCC. Cells were maintained in DMEM (HeLa cells) or RPMI 1640 (INCL lymphoblasts and T24 cells) supplemented with penicillin (50 units/mL), streptomycin (50 μg/mL) and 10% FBS (HeLa cells and T24 cells) or 15% FBS (INCL Lymphoblasts) at 37° C., 5% CO₂.

Plasmid Construction

mEGFP-HRas was a gift from Karel Svoboda² (Addgene plasmid #18662), mEGFP-N1 was a gift from Michael Davidson (Addgene plasmid #54767) and Hs.KRAS4b was a gift from Dominic Esposito (Addgene plasmid #83129). EGFP-KRas4b was constructed using Gibson Assembly (New England Biolabs). The vector mEGFP-N1 was linearized using HindIll and inserts were prepared by PCR amplification of Hs.KRAS4b with the following primers: KRAS4bfwd, 5′-GGA-CTC-AGA-TCT-CGA-GCT-CAA-ATG-ACT-GAA-TAT-AAA-CTT-GTG-G-3′; KRAS4brev, 5′-CCG-TCG-ACT-GCA-GAA-TTC-GAT-TAC-ATA-ATT-ACA-CAC-TTT-GTC-TTT-G-3′. The resulting construct was sequenced to verify its identity. Plasmids used for transfection were prepared using a plasmid maxiprep kit (EZgene).

Live-Cell Imaging of HeLa Cells

HeLa cells were plated at 40,000 cells/well in an 8-well Lab-Tek chamber slide (ThermoFisher) and allowed to adhere overnight. Cells were transfected with mEGFP-HRas or mEGFP-KRas4b using Lipofectamine 2000 (ThermoFisher) according to the manufacturer's protocol. 50 mM stocks of compounds 1 and 2 were prepared by dissolving the solid compound in DMSO containing 100 mM tris(2-S 3 carboxyethyl)phosphine hydrochloride (TCEP) as a preservative. From these stocks, a solution of 200 μM [1 or 2] (with 400 μM TCEP), or 400 μM TCEP was prepared in OptiMEM media. Before imaging, cells were exchanged into OptiMEM media. The diluted solutions were added to the indicated final concentration within individual wells of the chamber slide and cells imaged while maintaining 37° C., 5% CO₂ in the incubation chamber. Images were acquired in 6 different locations across a minimum of 2 independent experiments. The percentage of cells exhibiting GFP fluorescence at the plasma membrane before and after treatment in each location was counted. An unpaired t test was performed to determine the significance of the means before and after treatment.

Acyl Resin-Assisted Capture Detection of Protein Palmitoylation in HeLa Cells

HeLa cells were grown to confluency in 10 cm plates. Stock solutions of compounds were prepared in DMSO as before and diluted to the final indicated concentration in 5.5 mL of OptiMEM media before adding to cells. Cells were incubated at 37° C. for 20 min and then media removed and 3 mL of HBSS added to each plate. Cells were detached using a cell scraper, pelleted by centrifugation at 1,000 rcf for 5 min and the pellet processed using a CAPTUREome S-Palmitoylated Protein Kit (Badrilla, UK) according to the manufacturer's protocol. Samples were resolved by electrophoresis using 4-20% SDS-polyacrylamide gels (Bio-Rad) under denaturing and reducing conditions. Proteins were then electrotransferred to a PVDF membrane (Bio-Rad). The membrane was blocked with 3% BSA and then subjected to immunoblot analysis using Anti-GTPase HRAS antibody (ab97488) (Abcam), Calnexin Antibody (2433S) (Cell Signaling Technology), Gα(i) Antibody (5290S) (Cell Signaling Technology), SNAP25 Antibody (5308S) (Cell Signaling Technology), Flotillin-2 Antibody (3436S) (Cell Signaling Technology), CD44 Antibody (3570S) (Cell Signaling Technology), Lyric/Metadherin Antibody (14065S) (Cell Signaling Technology), Flotillin-1 Antibody (3253S) (Cell Signaling Technology) and secondary goat anti-rabbit IgG-HRP (sc-2030) (Santa Cruz Biotechnology) or goat anti-mouse IgG HRP (ThermoFisher). Chemiluminescent detection was performed by using SuperSignal West Pico PLUS chemiluminescent substrate (ThermoFisher) according to the manufacturer's instructions.

Acyl Resin-Assisted Capture Detection of GAP43 Palmitoylation in INCL Lymphoblasts

INCL Lymphoblasts were adjusted to 1,000,000 cells/mL in 20 mL of OptiMem Media. Stock solutions of compounds were prepared in DMSO as before and diluted to the indicated final concentration in the cell suspension. Cells were transferred to T25 flasks and incubated at 37° C. for 20 min. Cells were pelleted by centrifugation at 1,000 rcf for 5 min and the pellet processed using a CAPTUREome S-Palmitoylated Protein Kit (Badrilla, UK) according to the manufacturer's protocol. Samples were resolved by electrophoresis using 4-20% SDS-polyacrylamide gels (Bio-Rad) under denaturing and reducing conditions. Proteins were then electrotransferred to a PVDF membrane (Bio-Rad). The membrane was blocked with 3% BSA and then subjected to immunoblot analysis using GAP-43 (B-5) Antibody (sc-17790) (Santa Cruz Biotechnology) and secondary antibody goat anti-mouse IgG HRP (ThermoFisher). Chemiluminescent detection was performed by using SuperSignal West Pico PLUS chemiluminescent substrate (ThermoFisher) according to the manufacturer's instructions.

Statistical Analysis of Acyl Resin-Assisted Capture Detection Assays

Assays were performed in 3 biological replicates. Each replicate involved the analysis of 4 to 5 conditions side-by-side. Western blots were analyzed using ImageJ¹. The intensity of the protein band in each cleaved bound fraction (palmitoylated protein fraction) was normalized to the intensity of the corresponding input fraction (total protein fraction). The values within individual S 4 replicates were normalized to the control or vehicle condition and reported as means±SD. An unpaired t test was performed to determine the significance between means.

Detection of AKT1 and ERK1/2 Phosphorylation in HeLa Cells

HeLa cells were grown to confluency in 6 cm plates and then serum starved overnight in OptiMEM. Stock solutions of compounds were prepared in DMSO as before and diluted to the final indicated concentration in 2 mL of OptiMEM media before adding to cells. Cells were incubated at 37° C. for 20 min and then stimulated with 100 ng/μL EGF (ThermoFisher) for 5 min. Media was then removed and cells washed once with HBSS. 1.5 mL of HBSS was then added to each plate. Cells were detached using a cell scraper, pelleted by centrifugation at 1,000 rcf for 5 min and the pellet processed using MPER™ Mammalian Protein Extraction Reagent (ThermoFisher) with the addition of Halt Protease Inhibitor Cocktail (ThermoFisher) and 1 mM sodium orthovanadate. Samples were resolved by electrophoresis using 4-20% SDS-polyacrylamide gels (Bio-Rad) under denaturing and reducing conditions. Proteins were then electrotransferred to a PVDF membrane (Bio-Rad). The membrane was blocked with 3% BSA and then subjected to immunoblot analysis using AKT/MAPK Signaling Pathway Antibody Cocktail (ab151279) (Abcam) and secondary goat anti-rabbit IgG-HRP (sc-2030) (Santa Cruz Biotechnology). Chemiluminescent detection was performed by using SuperSignal West Pico PLUS chemiluminescent substrate (ThermoFisher) according to the manufacturer's instructions.

Statistical Analysis of AKT1 and ERK1/2 Phosphorylation in HeLa Cells

Assays were performed in 3 biological replicates. Western blots were analyzed using ImageJl. The intensity of the protein band in each condition was normalized to the intensity of the corresponding loading control (Rab11). The values within individual replicates were normalized to the vehicle condition and reported as means±SD. An unpaired t test was performed to determine the significance between means.

Detection of AKT1 and ERK1/2 Phosphorylation in T24 Cells

T24 cells were grown to confluency in 6 cm plates. Stock solutions of compounds were prepared in DMSO as before and diluted to the final indicated concentration in 2 mL of OptiMEM media before adding to cells. Cells were incubated at 37° C. for 1 h and then media removed and 1.5 mL of HBSS added to each plate. Cells were detached using a cell scraper, pelleted by centrifugation at 1,000 rcf for 5 min and the pellet processed using MPER™ Mammalian Protein Extraction Reagent (ThermoFisher) with the addition of Halt Protease Inhibitor Cocktail (ThermoFisher) and 1 mM sodium orthovanadate. Samples were resolved by electrophoresis using 4-20% SDS-polyacrylamide gels (Bio-Rad) under denaturing and reducing conditions. Proteins were then electrotransferred to a PVDF membrane (Bio-Rad). The membrane was blocked with 3% BSA and then subjected to immunoblot analysis using AKT/MAPK Signaling Pathway Antibody Cocktail (ab151279) (Abcam) and secondary goat anti-rabbit IgG-HRP (sc-2030) (Santa Cruz Biotechnology). Chemiluminescent detection was performed by using SuperSignal West Pico PLUS chemiluminescent substrate (ThermoFisher) according to the manufacturer's instructions.

Statistical Analysis of AKT1 and ERK1/2 Phosphorylation in T24 Cells

Assays were performed in 3 biological replicates. Western blots were analyzed using ImageJl. The intensity of the protein band in each condition was normalized to the intensity of the corresponding loading control (Rab11). The values within individual replicates were normalized to the vehicle condition and reported as means±SD.

WST-1 Viability Assay INCL Lymphoblasts

INCL lymphoblasts were plated at 100,000 cells/well in 80 μL of OptiMEM in a 96 well plate. Stock organic solutions were prepared as before and then diluted to 5× stock solutions in OptiMEM. 20 μL of the OptiMEM stocks was then added to the appropriate wells to achieve the indicated final concentration of the compound in 100 μL of cell suspension. After a 24 h incubation, 10 μL of WST-1 reagent (Sigma-Aldrich) was added to each well and cells incubated for 1 h at 37° C., 5% CO₂. Absorbance measurements were taken using a Satire II plate reader (Tecan) at 440 nm using 690 nm as a reference. The background absorbance of the WST-1 reagent in media was subtracted and cell viability was reported as a percentage of the viability of the non-treated control cells. All conditions were tested in 4 replicate wells and values reported as means±SD.

MS Detection of AMD Product

HeLa cells were grown to confluency in two 6 cm plates. Once confluent, media was removed and cells washed 1× with HBSS. Stock solutions were prepared as before. From these stocks, solutions of 200 μM 1 (with 400 μM TCEP) or 400 μM TCEP were prepared in OptiMEM media. To each plate was added 3 mL OptiMEM+400 μM TCEP or OptiMEM+200 μM 1 (with 400 μM TCEP). Cells were incubated at 37° C. for 20 min and then media removed and 3 mL of HBSS added to each plate. Cells were detached using a cell scraper, pelleted by centrifugation at 1,000 rcf for 5 min and the pellet subjected to a lipid extraction using the Bligh and Dyer method.³ Lipid extracts were analyzed for the presence of the AMD product palmityl-N-L-Cys-Oct (4) using reverse phase chromatography on an HP 1100 LC station to a Thermo LCQdeca-MS running in ESI Positive Ion Mode. MS/MS analysis was performed on the [M+H]⁺ peak of 4 (m/z 471) then selected reaction monitoring on the fragmentation peak (m/z 130) (FIG. 9).

Synthesis

General Considerations

Commercially available N-Boc-L-Cys(Trt)-OH, O-(7-azabenzotriazol-1-yl)-1,1,3,3-tetramethyluronium hexafluorophosphate (HATU), N,N-diisopropylethylamine (DIEA), octylamine, trifluoroacetic acid (TFA), triethylsilane (TES), dimethyl sulfoxide (DMSO), tris(2-carboxyethyl)phosphine hydrochloride (TCEP.HCl) and N-tert-butylhydroxylamine hydrochloride (NtBuHA) were obtained from Sigma-Aldrich. Deuterated chloroform (CDCl₃) and methanol (CD₃OD) were obtained from Cambridge Isotope Laboratories. All reagents obtained from commercial suppliers were used without further purification unless otherwise noted. Analytical thin-layer chromatography was performed on E. Merck silica gel 60 F₂₅₄ plates. Compounds, which were not UV active, were visualized by dipping the plates in a ninhydrin or potassium permanganate solution and heating. Silica gel flash chromatography was performed using E. Merck silica gel (type 60SDS, 230-400 mesh). Solvent mixtures for chromatography are reported as v/v ratios. HPLC analysis was carried out on an Eclipse Plus C8 analytical column with Phase AlPhase B gradients [Phase A: H₂O with 0.1% formic acid; Phase B: MeOH with 0.1% formic acid]. HPLC purification was carried out on Zorbax SB-C18 semipreparative column with Phase AlPhase B gradients [Phase A: H₂O with 0.1% formic acid; Phase B: MeOH with 0.1% formic acid]. Proton nuclear magnetic resonance (¹H NMR) spectra were recorded on a Varian VX-500 MHz or Jeol Delta ECA-500 MHz spectrometers, and were referenced relative to residual proton resonances in CDCl₃ (at δ 7.24 ppm) or CD₃OD (at δ 4.87 or 3.31 ppm). ¹H NMR splitting patterns are assigned as singlet (s), doublet (d), triplet (t), quartet (q) or pentuplet (p). All first-order splitting patterns were designated on the basis of the appearance of the multiplet. Splitting patterns that could not be readily interpreted are designated as multiplet (m) or broad (br). Carbon nuclear magnetic resonance (¹³C NMR) spectra were recorded on a Varian VX-500 MHz or Jeol Delta ECA-500 MHz spectrometers, and were referenced relative to residual proton resonances in CDCl₃ (at δ 77.23 ppm) or CD₃OD (at δ 49.15 ppm). Electrospray Ionization-Time of Flight (ESI-TOF) spectra were obtained on an Agilent 6230 Accurate-Mass TOFMS mass spectrometer.

Synthesis of Alkyl Reagents

N-Boc-L-Cys(Trt)-Oct.

A solution of N-Boc-L-Cys(Trt)-OH (250.0 mg, 539.3 μmop in CH₂Cl₂ (5 mL) was stirred at 0° C. for 10 min, and then HATU (225.5 mg, 593.2 μmop and DIEA (375.7 μL, 2.16 mmol) were successively added. After 10 min stirring at 0 octylamine (89.1 μL, 539.3 μmop was added. After 1 h stirring at rt, the reaction mixture was washed with HCl(5%) (3×2.5 mL) and NaHCO₃(sat) (3×2.5 mL). The organic layer was dried (Na₂SO₄), filtered and concentrated, providing a yellow oil, which was purified by flash chromatography (0-2% MeOH in CH₂Cl₂), affording 295.9 mg of N-Boc-L-Cys(Trt)-Oct as a pale yellow oil [95%, R_{f}=0.40 (1% MeOH in CH₂Cl₂)]. ¹H NMR (CDCl₃, 500.13 MHz, δ): 7.41 (d, J=8.0 Hz, 5H, 5×CH_{A}), 7.35-7.15 (m, 10H, 10×CH_{A}), 6.04-5.89 (m, 1H, 1×NH), 4.92-4.69 (m, 1H, 1×NH), 3.90-3.72 (m, 1H, 1×CH), 3.26-3.04 (m, 2H, 1×CH₂), 2.79-2.68 (m, 1H, 0.5×CH₂), 2.59-2.37 (m, 1H, 0.5×CH₂), 1.35-1.15 (m, 12H, 6×CH₂), 1.41 (s, 9H, 3×CH₃), 0.86 (t, J=6.9 Hz, 3H, 1×CH₃). ¹³C NMR (CDCl₃, 125.77 MHz, δ): 170.3, 155.5, 144.5, 129.7, 128.2, 127.0, 80.4, 67.3, 53.6, 39.6, 38.8, 31.9, 29.5, 29.4, 29.3, 28.4, 26.9, 22.8, 14.2. MS (ESI-TOF) [m/z (%)]: 597 ([M+Na]⁺, 100), 575 ([MH]⁺, 68]. HRMS (ESI-TOF) calculated for [C₃₅H₄₆N₂O₃SNa] ([M+Na]⁺) 597.3121, found 597.3124.

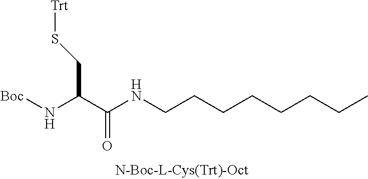

H₂N-L-Cys-Oct∪H₂N-L-Cys-Oct (1′).* * Air oxidation of the thiol (RSH) causes the disulfide bond (RS-SR) formation.

A solution of N-Boc-L-Cys(Trt)-Oct (10.0 mg, 17.4 μmol) in 500 μL of TFA/CH₂Cl₂/TES (225:225:50) was stirred at rt for 30 min After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 3.27 mg of the H₂N-L-Cys-Oct H₂N-L-Cys-Oct (1′) as a colorless film [81%, t_{R}=6.6 min (Zorbax SB-C18 semipreparative column, 50% Phase A in Phase B, 5 min, and then 5% Phase A in Phase B, 10 min)]. As a disulfide (RS-SR): t_{R}=2.52 min (Eclipse Plus C8 analytical column, 5% Phase A in Phase B, 5.5 min)] (FIG. 8). ¹H NMR (CD₃OD, 500.13 MHz, 5): 4.11-3.91 (m, 2H, 2×CH), 3.36-3.32 (m, 2H, 2×CH), 3.29-3.21 (m, 4H, 2×CH₂), 3.09-2.92 (m, 2H, 2×CH), 1.65-1.47 (m, 4H, 2×CH₂), 1.45-1.21 (m, 20H, 10×CH₂), 0.91 (t, J=6.8 Hz, 6H, 2×CH₃). ¹³C NMR (CD₃OD, 125.77 MHz, 5): 170.2, 53.6, 40.9, 40.8, 33.1, 30.5, 30.4, 30.3, 28.1, 23.8, 14.5. MS (ESI-TOF) [m/z (%)]: 485 ([M+Na]⁺, 100), 463 ([MH]⁺, 33]. HRMS (ESI-TOF) calculated for [C₂₂H₄₆N₄O₂S₂Na] ([M+Na]⁺) 485.2954, found 485.2947. HRMS (ESI-TOF) calculated for [C₂₂H₄₇N₄O₂S₂] ([MH]⁺) 463.3135, found 463.3128.

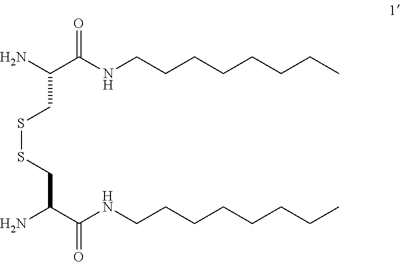

H₂N-L-Cys-Oct (1).

A solution of H₂N-L-Cys-Oct U H₂N-L-Cys-Oct (1′, 1.0 mg, 4.3 μmol) in 861.5 μL of a 5 mM solution of TCEP.HCl in H₂O (or DMSO) was stirred at rt for 5 min. Then, it was analyzed by HPLC and/or used directly for the depalmitoylation experiments. As a free thiol (R-SH): t_{R}=2.77 min (Eclipse Plus C8 analytical column, 5% Phase A in Phase B, 5.5 min)] (FIG. 8). MS (ESI-TOF) [m/z (%)]: 233 ([MH]⁺, 100).

N-Boc-L-Ser(^{t}Bu)-Oct.

A solution of N-Boc-L-Ser(Bu)-OH (100.0 mg, 382.8 μmol) in CH₂Cl₂ (4 mL) was stirred at 0° C. for 10 min, and then HATU (160.1 mg, 421.1 μmol) and DIEA (266.7 μL, 1.53 mmol) were successively added. After 10 min stirring at 0 octylamine (63.3 μLm 382.8 μmol) was added. After 1 h stirring at rt, the reaction mixture was washed with HCl(5%) (3×2 mL) and NaHCO₃(sat) (3×2 mL). The organic layer was dried (Na₂SO₄), filtered and concentrated, providing a yellow oil, which was purified by flash chromatography (0-5% MeOH in CH₂Cl₂), affording 128.9 mg of N-Boc-L-Ser(^{t}Bu)-Oct as white crystals [91%, R_{f}=0.42 (5% MeOH in CH₂Cl₂)]. ¹H NMR (CDCl₃, 500.13 MHz, δ): 6.69-6.41 (m, 1H, 1×NH), 5.54-5.31 (m, 1H, 1×NH), 4.21-4.00 (m, 1H, 1×CH), 3.86-3.66 (m, 1H, 0.5×CH₂), 3.43-3.29 (m, 1H, 0.5×CH₂), 3.29-3.19 (m, 2H, 1×CH₂), 1.51-1.46 (m, 2H, 1×CH₂), 1.44 (s, 9H, 3×CH₃), 1.34-1.20 (m, 10H, 5×CH₂), 1.18 (s, 9H, 3×CH₃), 0.86 (t, J=6.9 Hz, 3H, 1×CH₃). ¹³C NMR (CDCl₃, 125.77 MHz, δ): 170.6, 155.7, 80.0, 74.0, 62.0, 54.3, 39.6, 38.8, 31.9, 29.6, 29.4, 28.5, 27.6, 27.0, 22.8, 14.2. MS (ESI-TOF) [m/z (%)]: 373 ([MH]⁺, 100]. HRMS (ESI-TOF) calculated for [C₂₀H₄₁N₂O₄] ([MH]⁺) 373.3061, found 373.3058.

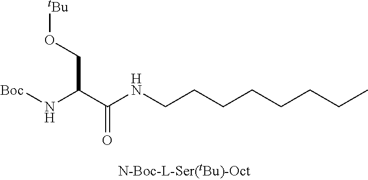 [SMILES: CCCCCCCCNC(=O)[C@H](COC(C)(C)C)NC(=O)OC(C)(C)C]

H₂N-L-Ser-Oct (2).

A solution of N-Boc-L-Ser(tBu)-Oct (20.0 mg, 53.7 μmop in 500 pt of TFA/CH₂Cl₂/TES (225:225:50) was stirred at rt for 30 min After removal of the solvent, the residue was dried under high vacuum for 3 h. Then, the corresponding residue was diluted in MeOH (250 μL), filtered using a 0.2 μm syringe-driven filter, and the crude solution was purified by HPLC, affording 10.1 mg of the H₂N-L-Ser-Oct (2) as a colorless film [87%, t_{R}=6.3 min (Zorbax SB-C18 semipreparative column, 50% Phase A in Phase B, 5 min, and then 5% Phase A in Phase B, 10 min)]. ¹H NMR (CD₃OD, 500.13 MHz, δ): 3.92-3.87 (m, 1H, 1×CH), 3.86-3.81 (m, 1H, 0.5×CH₂), 3.80-3.74 (m, 1H, 0.5×CH₂), 3.26-3.22 (t, J=7.0 Hz, 2H, 1×CH₂), 1.59-1.47 (m, 2H, 1×CH₂), 1.36-1.23 (m, 10H, 5×CH₂), 0.90 (t, J=7.0 Hz, 3H, 1×CH₃). ¹³C NMR (CD₃OD, 125.77 MHz, δ): 168.4, 62.0, 56.5, 40.7, 33.0, 30.4, 30.4, 30.3, 28.0, 23.7, 14.4. MS (ESI-TOF) [m/z (%)]: 217 ([MH]⁺, 100]. HRMS (ESI-TOF) calculated for [C₁₁H₂₅N₂O₂] ([MH]⁺) 217.1911, found 217.1912.

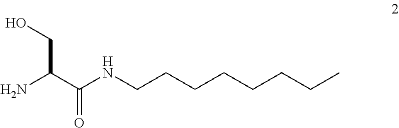

Synthesis of Thioesters

MESNA Thiopalmitate (3).⁴

A solution of palmitic acid (171.8 mg, 670.0 μmol) in CH₂Cl₂ (5 mL) was stirred at 0° C. for 10 min, and then DMAP (7.4 mg, 60.9 μmol) and EDC.HCl (128.4 mg, 670.0 μmol) were successively added. After 10 min stirring at 0° C., sodium 2-mercaptoethanesulfonate⁵ (MESNA, 100.0 mg, 609.1 μmol) was added. After 5 h stirring at rt, the mixture was extracted with H₂O (2×3 mL) and the combined aqueous phases were washed with EtOAc (3 mL). After evaporation of H₂O under reduced pressure, the residue was washed with CH₃CN (5 mL), and then filtered to yield 189.3 mg of 3 as a white solid [77%]. ¹H NMR (d₆-DMSO, 500.13 MHz, δ): 3.08-2.96 (m, 2H, 1×CH₂), 2.62-2.50 (m, 4H, 2×CH₂), 1.62-1.45 (m, 2H, 1×CH₂), 1.34-1.14 (m, 24H, 12×CH₂), 0.85 (t, J=7.0 Hz, 3H, 1×CH₃). ¹³C NMR (d₆-DMSO, 125.77 MHz, δ): 198.7, 50.9, 43.3, 31.3, 29.1, 29.1, 29.1, 29.0, 29.0, 29.0, 28.9, 28.8, 28.7, 28.2, 25.1, 24.3, 22.1, 14.0. MS (ESI-TOF) [m/z (%)]: 379 ([M-Na]⁻, 100). HRMS (ESI-TOF) calculated for C₁₈H₃₅O₄S₂ ([M-Na]⁻) 379.1971, found 379.1973.

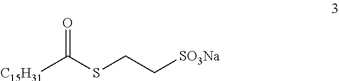

Synthesis of Lipids

Palmityl-N-L-Cys-Oct (4).

H₂N-L-Cys-Oct (1, 3.00 mg, 12.93 μmol) and MESNA thiopalmitate (3, 5.20 mg, 12.93 μmol) were dissolved in 1.29 mL of 20 mM TCEP.HCl in 200 mM NaH₂PO₄ pH 7.1 buffer and stirred under N₂ at rt. After 30 min, the corresponding mixture was filtered using a 0.2 μm syringe-driven filter, and the crude solution was purified by HPLC, affording 4.92 mg of the amidophospholipid 4 as a colorless oil [81%, t_{R}=8.1 min (Zorbax SB-C18 semipreparative column, 100% Phase B, 20.5 min), t_{R}=5.01 min (Eclipse Plus C8 analytical column, 5% Phase A in Phase B, 5.5 min)]. MS (ESI-TOF) [m/z (%)]: 493 ([M 18), 471 ([MH]⁺, 100]. HRMS (ESI-TOF) calculated for [C₂₇H₅₅N₂O₂S] ([MH]⁺) 471.3984, found 471.3982.

### Example 2: Neuronal Ceroid Lipofuscinoses

Neuronal ceroid lipofuscinoses (NCLs) are commonly grouped together as Batten disease. NCLs are the most common neurodegenerative lysosomal storage diseases (LSDs) of the pediatric population. Batten disease is rare (1 per 12,500 births), inherited, and neurodegenerative. Symptoms include progressive intellectual and motor deterioration, seizures, and early death. Visual loss is also a feature of most forms. Phenotypes include congenital, infantile (INCL), late-infantile (LINCL), juvenile (JNCL), northern epilepsy (NE), and adult (ANCL; Kufs or Parry diseases). INCL is a CLN1 disorder, which has been genetically mapped to PPT1, and with an onset of symptoms between 6-24 months of age.

### Example 3: Prodrug Strategy for In Vivo Depalmitoylation of Proteins

We demonstrated that the prodrug compound was capable of depalmitoylating proteins in vivo. Administration of the compound to cells expressing the palmitoylated protein HRas-GFP resulted in the release of the protein from the plasma membrane, indicative of depalmitoylation of said protein.

The present disclosure describes an alkyl cysteine prodrug which, upon cleavage by endogenous esterases, removes S-palmitoyl groups on native proteins via native chemical ligation (ncl).

The compounds provided herein including embodiments thereof exhibit good resistance to oxidative deactivation due to the methyl acetate substituted thiol. This allows for the storage and administration of this compound without the need for a reducing agent. This substitution may also enhance bioavailability and delivery in vivo.

The thiol of alkyl cysteines may be substituted with a methyl acetate group (a thiol protecting group). This group prevents oxidation of the thiol and may improve the bioavailability and cellular delivery. Upon entering the cell, the acetyl group is cleaved and the free thiol is exposed, forming the active depalmitoylation agent. We have validated the activity in live cell assays and the compounds provided herein may be useful for therapeutic applications in diseases in which palmitoylation is dysregulated, e.g., cancer, Alzheimer's, or Infantile Neuronal Ceroid Lipofuscinosis.

### P1 EMBODIMENTS

P1 Embodiment 1. A method of treating neuronal ceroid lipofuscinoses in a subject in need thereof, the method comprising administering an effective amount of a depalmitoylation agent.

P1 Embodiment 2. The method of embodiment 1, wherein said depalmitoylation agent is an alkyl cysteine or a sulfhythyl-protected alkyl cysteine.

P1 Embodiment 3. The method of embodiment 1, wherein said sulfhydryl-protected alkyl cysteine has the formula:

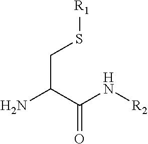

Wherein R¹ is a sulfhydryl protecting group and R² is a substituted or unsubstituted alkyl or a substituted or unsubstituted heteroalkyl.

P1 Embodiment 4. The method of embodiment 3, wherein R² is an unsubstituted C₁-C₂₀ alkyl.

P1 Embodiment 5. The method of embodiment 3 or 4, wherein R² is an unsubstituted C₁-C₁₀ alkyl.

P1 Embodiment 6. The method of any one of embodiments 3-5, wherein R² is an unsubstituted C₄-C₈ alkyl.

P1 Embodiment 7. The method of any one of embodiments 3-6, wherein R¹ is a light labile sulfhydryl protecting group.

P1 Embodiment 8. The method of any one of embodiments 3-7, wherein R¹ is

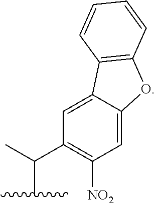 [SMILES: *C(C)c1cc2c(cc1[N+](=O)[O-])oc1ccccc12]

### P2 EMBODIMENTS

P2 Embodiment 1. A depalmitoylating prodrug compound substantially as hereinbefore described with reference to any one of the Examples or to any one of the accompanying drawings.

P2 Embodiment 2. A method of treating a disease caused by palmitoylation dysregulation in a subject in need thereof, the method comprising administering to said subject an effective amount of the depalmitoylating prodrug compound of embodiment 1.

P2 Embodiment 3. The method of embodiment 2, wherein the disease is cancer, Alzheimer's disease or Infantile Neuronal Ceroid Lipofuscinosis.

### EMBODIMENTS

Embodiment 1. A compound of formula:

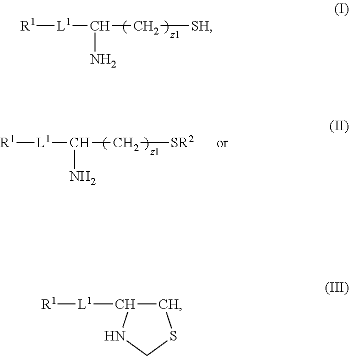

wherein
   R¹ is hydrogen, —N(R⁴)(R⁵), —NjR⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is a thiol protecting group;
   R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
      z1 is an integer from 0 to 5.

Embodiment 2. The compound of embodiment 1, wherein R¹ is —N(R⁴)(R⁵), —N(R⁴)(R⁵)(R⁶), substituted or unsubstituted C₁-C₂₅ alkyl, or substituted or unsubstituted aryl.

Embodiment 3. The compound of embodiment 1 or 2, wherein R¹ is —N(R⁴)(R⁵) and R⁴ and R⁵ are independently unsubstituted C₁-C₁₀ alkyl.

Embodiment 4. The compound of any one of embodiments 1-3 wherein R⁴ and R⁵ are independently unsubstituted C₁ alkyl.

Embodiment 5. The compound of embodiment 1 or 2, wherein R¹ is —N(R⁴)(R⁵)(R⁶) and R⁴, R⁵ and R⁶ are independently unsubstituted C₁-C₁₀ alkyl.

Embodiment 6. The compound of any one of embodiments 1, 2, or 5, wherein R⁴, R⁵ and R⁶ are independently unsubstituted C₁ alkyl.

Embodiment 7. The compound of embodiment 1 or 2, wherein R¹ is unsubstituted C₁-C₂₅ alkyl.

Embodiment 8. The compound of any one of embodiments 1, 12 or 7, wherein R¹ is unsubstituted C₁-C₂₅ alkyl.

Embodiment 9. The compound of any one of embodiments 1, 2 or 8, wherein R¹ is unsubstituted C₈ alkyl.

Embodiment 10. The compound of embodiment 1 or 2, wherein, R¹ is R^{1A}-substituted C₁-C₂₅
wherein
   R^{1A} is independently hydrogen, —N(R^{4A})(R^{5A}), —N⁺(R^{4A})(R^{5A})(R^{6A}), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
      R^{4A}, R^{5A} and R^{6A} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 11. The compound of embodiment 10, wherein R^{1A} is —N(R^{4A})(R^{5A}) or —N⁺(R^{4A})(R^{5A})(R^{6A}) and R^{4A}, R^{5A} and R^{6A} are independently substituted or unsubstituted C₁-C₅ alkyl.

Embodiment 12. The compound of embodiment 10 or 11, wherein R^{4A}, R^{5A} and R^{6A} are independently unsubstituted C₁ alkyl.

Embodiment 13. The compound of embodiment 10, wherein R^{1A} is unsubstituted 5-10-membered aryl.

Embodiment 14. The compound of embodiment 10 or 13, wherein R^{1A} is unsubstituted phenyl or unsubstituted naphthyl.

Embodiment 15. The compound of embodiment 1 or 2, wherein R¹ is substituted or unsubstituted 5-10-membered aryl.

Embodiment 16. The compound of any one of embodiments 1, 2 or 15 wherein R¹ is substituted or unsubstituted phenyl.

Embodiment 17. The compound of any one of embodiments 1, 2, 15 or 16, wherein R¹ is unsubstituted phenyl.

Embodiment 18. The compound of any one of embodiments 1, 2, or 15, wherein R¹ is substituted or unsubstituted naphthyl.

Embodiment 19. The compound of any one of embodiments 1, 2, 15 or 18, wherein R¹ is unsubstituted naphthyl.

Embodiment 20. The compound of any one of embodiments 1, 2, or 15, wherein R¹ is R^{1A}-substituted 5-10-membered aryl, wherein R^{1A} is unsubstituted C₁-C₂₅

Embodiment 21. The compound of embodiment 20, wherein R¹ is R^{1A}-substituted phenyl and R^{1A} is unsubstituted C₈ alkyl.

Embodiment 22. The compound of any one of embodiments 1-21, wherein R² is —SR³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein
   R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 23. The compound of any one of embodiments 1-22, wherein R² is —SR³ or substituted or unsubstituted heteroalkyl.

Embodiment 24. The compound of any one of embodiments 1-23, wherein R² is substituted 2-8 membered heteroalkyl.

Embodiment 25. The compound of any one of embodiments 1-24, wherein R² is substituted 4 membered heteroalkyl.

Embodiment 26. The compound of any one of embodiments 1-25, wherein R² is

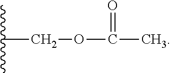 [SMILES: *COC(C)=O]

Embodiment 27. The compound of any one of embodiments 1-23, wherein R² is —SR³ and R³ is substituted or unsubstituted C₁-C₅ alkyl.

Embodiment 28. The compound of embodiment 27, wherein R³ is unsubstituted C₁-C₁₂ alkyl.

Embodiment 29. The compound of any one of embodiments 1-23, wherein R² is —SR³ and R³ is substituted or unsubstituted C₅-C₁₀ aryl.

Embodiment 30. The compound of embodiment 29, wherein R³ is unsubstituted phenyl.

Embodiment 31. The compound of any one of embodiments 1-23, wherein R² is —SR³ and R³ is substituted or unsubstituted 5 to 10 membered heteroaryl.

Embodiment 32. The compound of embodiment 31, wherein R³ is unsubstituted pyridyl.

Embodiment 33. The compound of any one of embodiments 1-32, wherein L¹ is a bond, substituted or unsubstituted alkylene or

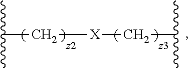

wherein
   X is a bond, —S—, —O—, —NH—, —C(O)—NH— or —C(O)—; and
      z2 and z3 are independently integers from 0 to 25.

Embodiment 34. The compound of embodiment 33, wherein X is —C(O)—NH—.

Embodiment 35. The compound of any one of embodiments 1-34, wherein z11 or 2.

Embodiment 36. The compound of any one of embodiments 33-35, wherein z2 is 0 or 1.

Embodiment 37. The compound of any one of embodiments 33-36, wherein z3 is 0, 1, 2 or 4.

Embodiment 38. The compound of any one of embodiments 33-36, wherein z3 is an integer from 10-15.

Embodiment 39. The compound of any one of embodiments 1-33, wherein L¹ is a bond or unsubstituted C₁-C₈ alkylene.

Embodiment 40. The compound of any one of embodiments 1-33 or 39, wherein L¹ is unsubstituted C₂ alkylene or unsubstituted C₄ alkylene.

Embodiment 41. The compound of any one of embodiments 1-33 or 39-40, wherein L¹ is unsubstituted C₄ alkylene.

Embodiment 42. A pharmaceutical composition comprising a pharmaceutically acceptable excipient and a compound of any one of embodiments 1-41.

Embodiment 43. A method of treating a depalmitoylation-associated disease in a subject in need thereof, said method comprising administering to said subject a therapeutically effective amount of a compound of any one of embodiments 1-41, thereby treating a depalmitoylation-associated disease in said subject.

Embodiment 44. The method of embodiment 43, wherein said depalmitoylation-associated disease is cancer or a neurological disease.

Embodiment 45. The method of embodiment 43 or 44, wherein said depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis or glioma.

Embodiment 46. A method of depalmitoylating a protein in a cell comprising contacting said cell with an effective amount of a compound of any one of embodiments 1-41.

Embodiment 47. The method of embodiment 46, wherein said protein forms part of the plasma membrane of said cell.

Embodiment 48. The method of embodiment 46 or 47, wherein said protein is HRas, NRas, EGFR, amyloid precursor protein (APP), BACE1, EZH2, PD-L1, flotillin-1, flotillin-2, calnexin, Gα(i), metadherin, CD44 or SNAP25.

Embodiment 49. The method of any one of embodiments 46-48, wherein said contacting occurs in vitro or in vivo.

Embodiment 50. The method of any one of embodiments 46-49, wherein said cell forms part of an organism.

Embodiment 51. The method of any one of embodiments 46-50, wherein said cell forms part of a mammalian subject.

Embodiment 52. The method of embodiment 51, wherein said mammalian subject suffers from cancer or a neurological disease.

Embodiment 53. A method of treating a depalmitoylation-associated disease in a subject in need thereof, said method comprising administering to said subject a therapeutically effective amount of a depalmitoylating amphiphilic thiol compound, thereby treating a depalmitoylation-associated disease in said subject.

Embodiment 54. The method of embodiment 53, wherein said depalmitoylating amphiphilic thiol compound is a compound of any one of embodiments 1-41.

### ADDITIONAL EMBODIMENTS

Embodiment 1. A method of treating a neurological disease in a subject in need thereof, said method comprising administering to said subject a therapeutically effective amount of a compound of formula:

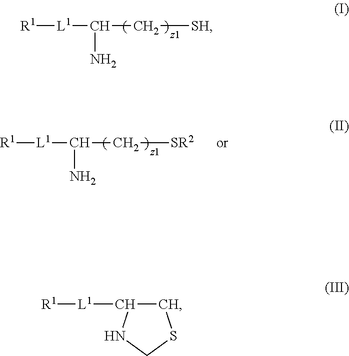

wherein
R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is a thiol protecting group;
R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and z1 is an integer from 0 to 5, thereby treating a neurological disease in said subject.

Embodiment 2. The method of embodiment 1, wherein said compound is:

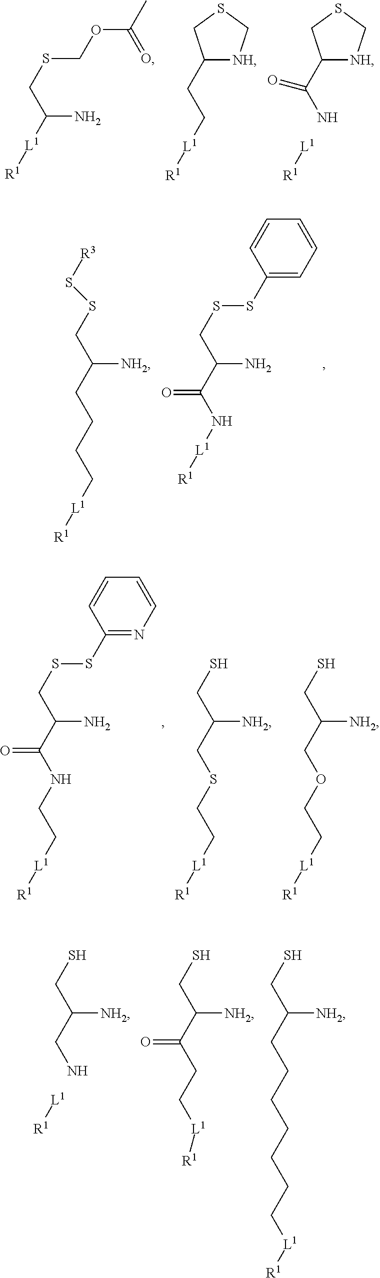

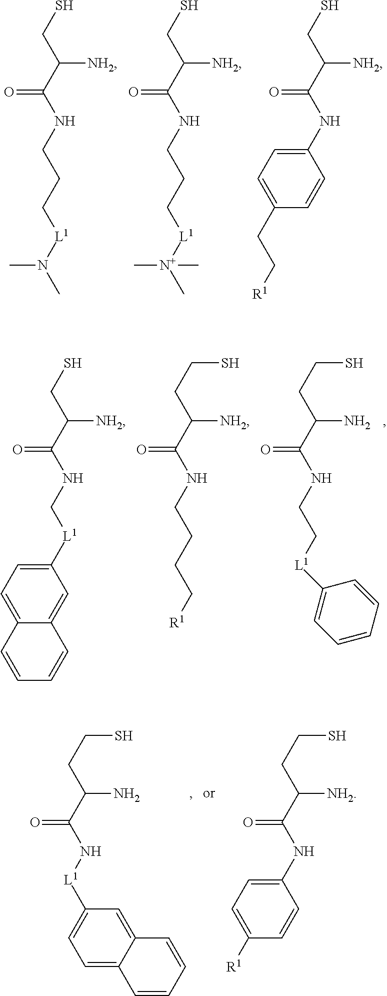

Embodiment 3. A method of treating cancer in a subject in need thereof, said method comprising administering to said subject a therapeutically effective amount of a compound of formula:

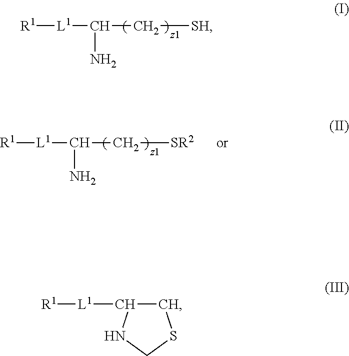

wherein
R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is a thiol protecting group;
R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
z1 is an integer from 0 to 5, thereby treating cancer in said subject.

Embodiment 4. The method of embodiment 4, wherein said compound is:

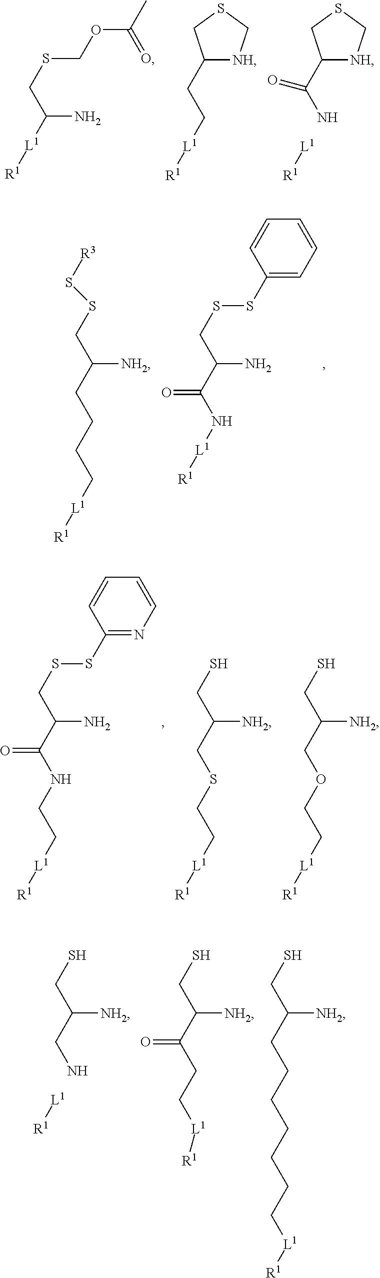

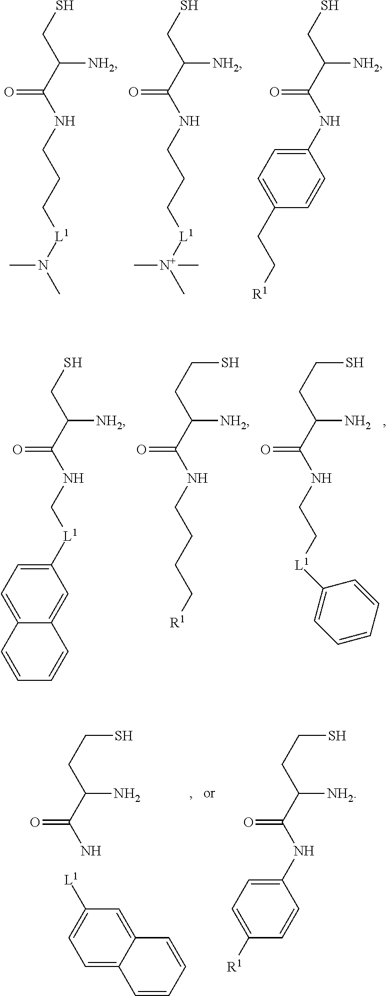

### REFERENCES

(1) Prabakaran, S.; Lippens, G.; Steen, H.; Gunawardena, J. Post-Translational Modification: Nature's Escape from Genetic Imprisonment and the Basis for Dynamic Information Encoding. Wiley Interdiscip. Rev. Syst. Biol. Med. 2012, 4, 565-583.
(2) Vidal, C. J. Post-Translational Modifications in Health and Disease; Springer New York: New York, N.Y., 2011.
(3) Yeste-Velasco, M.; Linder, M. E.; Lu, Y.-J. Protein S-Palmitoylation and Cancer. Biochim. Biophys. Acta 2015, 1856, 107-120.
(4) Rocks, O.; Gerauer, M.; Vartak, N.; Koch, S.; Huang, Z.-P.; Pechlivanis, M.; Kuhlmann, J.; Brunsveld, L.; Chandra, A.; Ellinger, B.; Waldmann, H.; Bastiaens, P. I. H. The Palmitoylation Machinery Is a Spatially Organizing System for Peripheral Membrane Proteins. Cell 2010, 141, 458-471.
(5) Meckler, X.; Roseman, J.; Das, P.; Cheng, H.; Pei, S.; Keat, M.; Kassarjian, B.; Golde, T. E.; Parent, A. T.; Thinakaran, G. Reduced Alzheimer's Disease B-Amyloid Deposition in Transgenic Mice Expressing S-Palmitoylation-Deficient APH1aL and Nicastrin. J. Neurosci. 2010, 30, 16160-16169.
(6) Berchtold, L. A.; Storling, Z. M.; Ortis, F.; Lage, K.; Bang-Berthelsen, C.; Bergholdt, R.; Hald, J.; Brorsson, C. A.; Eizirik, D. L.; Pociot, F.; Brunak, S.; Storling, J. Huntingtin-Interacting Protein 14 Is a Type 1 Diabetes Candidate Protein Regulating Insulin Secretion and B-Cell Apoptosis. Proc. Natl. Acad. Sci. 2011, 108, E681-E688.
(7) Blaskovic, S.; Blanc, M.; van der Goot, F. G. What Does S-Palmitoylation Do to Membrane Proteins? FEBS J. 2013, 280, 2766-2774.
(8) Dawson, P.; Muir, T.; Clark-Lewis, I.; Kent, S. Synthesis of Proteins by Native Chemical Ligation. Science 1994, 266, 776-779.
(9) Brea, R. J.; Cole, C. M.; Devaraj, N. K. In Situ Vesicle Formation by Native Chemical Ligation. Angew. Chem. Int. Ed. 2014, 53, 14102-14105.
(10) Brea, R. J.; Rudd, A. K.; Devaraj, N. K. Nonenzymatic Biomimetic Remodeling of Phospholipids in Synthetic Liposomes. Proc. Natl. Acad. Sci. 2016, 113, 8589-8594.
(11) Brea, R. J.; Cole, C. M.; Lyda, B. R.; Ye, L.; Prosser, R. S.; Sunahara, R. K.; Devaraj, N. K. In Situ Reconstitution of the Adenosine A2A Receptor in Spontaneously Formed Synthetic Liposomes. J. Am. Chem. Soc. 2017, 139, 3607-3610.
(12) Yan, L. Z.; Dawson, P. E. Synthesis of Peptides and Proteins without Cysteine Residues by Native Chemical Ligation Combined with Desulfurization. J. Am. Chem. Soc. 2001, 123, 526-533.
(13) Malumbres, M.; Barbacid, M. RAS Oncogenes: The First 30 Years. Nat. Rev. Cancer 2003, 3,459-465.
(14) Fernandez-Medarde, A.; Santos, E. Ras in Cancer and Developmental Diseases. Genes Cancer 2011, 2, 344-358. [0411]
(15) Aoki, Y.; Niihori, T.; Kawame, H.; Kurosawa, K.; Ohashi, H.; Tanaka, Y.; Filocamo, M.; Kato, K.; Suzuki, Y.; Kure, S.; Matsubara, Y. Germline Mutations in HRAS Proto-Oncogene Cause Costello Syndrome. Nat. Genet. 2005, 37, 1038-1040.
(16) Gripp, K. W.; Lin, A. E. Costello Syndrome: A Ras/Mitogen Activated Protein Kinase Pathway Syndrome (Rasopathy) Resulting from HRAS Germline Mutations. Genet. Med. 2012, 14, 285-292.
(17) Papke, B.; Der, C. J. Drugging RAS: Know the Enemy. Science 2017, 355, 1158-1163.
(18) Calvo, F.; Agudo-Ibáñez, L.; Crespo, P. The Ras-ERK Pathway: Understanding Site-Specific Signaling Provides Hope of New Anti-Tumor Therapies. BioEssays 2010, 32, 412-421.
(19) Goodwin, J. S.; Drake, K. R.; Rogers, C.; Wright, L.; Lippincott-Schwartz, J.; Philips, M. R.; Kenworthy, A. K. Depalmitoylated Ras Traffics to and from the Golgi Complex via a Nonvesicular Pathway. J. Cell Biol. 2005, 170, 261-272.
(20) Forrester, M. T.; Hess, D. T.; Thompson, J. W.; Hultman, R.; Moseley, M. A.; Stamler, J. S.; Casey, P. J. Site-Specific Analysis of Protein S-Acylation by Resin-Assisted Capture. J. Lipid Res. 2011, 52, 393-398.
(21) Agudo-Ibáñez, L.; Herrero, A.; Barbacid, M.; Crespo, P. H-Ras Distribution and Signaling in Plasma Membrane Microdomains Are Regulated by Acylation and Deacylation Events. Mol. Cell. Biol. 2015, 35, 1898-1914.
(22) Rusch, M.; Zimmermann, T. J.; Burger, M.; Dekker, F. J.; Gormer, K.; Triola, G.; Brockmeyer, A.; Janning, P.; Böttcher, T.; Sieber, S. A.; Vetter, I. R.; Hedberg, C.; Waldmann, H. Identification of Acyl Protein Thioesterases 1 and 2 as the Cellular Targets of the Ras-Signaling Modulators Palmostatin B and M. Angew. Chemie Int. Ed. 2011, 50, 9838-9842.
(23) Dekker, F. J.; Rocks, O.; Vartak, N.; Menninger, S.; Hedberg, C.; Balamurugan, R.; Wetzel, S.; Renner, S.; Gerauer, M.; Scholermann, B.; Rusch, M.; Kramer, J. W.; Rauh, D.; Coates, G. W.; Brunsveld, L.; Bastiaens, P. I. H.; Waldmann, H. Small-Molecule Inhibition of APT1 Affects Ras Localization and Signaling. Nat. Chem. Biol. 2010, 6, 449-456.
(24) Babuke, T.; Tikkanen, R. Dissecting the Molecular Function of Reggie/Flotillin Proteins. Eur. J. Cell Biol. 2007, 86, 525-532.
(25) Rajagopalan, S.; Xu, Y.; Brenner, M. B. Retention of Unassembled Components of Integral Membrane Proteins by Calnexin. Science 1994, 263, 387-390.
(26) De Luca, A.; Maiello, M. R.; D'Alessio, A.; Pergameno, M.; Normanno, N. The RAS/RAF/MEK/ERK and the PI3K/AKT Signalling Pathways: Role in Cancer Pathogenesis and Implications for Therapeutic Approaches. Expert Opin. Ther. Targets 2012, 16, S17-S27.
(27) Reddy, E. P.; Reynolds, R. K.; Santos, E.; Barbacid, M. A Point Mutation Is Responsible for the Acquisition of Transforming Properties by the T24 Human Bladder Carcinoma Oncogene. Nature 1982, 300, 149-152.
(28) Kiessling, M. K.; Curioni-Fontecedro, A.; Samaras, P.; Atrott, K.; Cosin-Roger, J.; Lang, S.; Scharl, M.; Rogler, G. Mutant HRAS as Novel Target for MEK and MTOR Inhibitors. Oncotarget 2015, 6, 42183-42196.
(29) Sanders, S. S.; Martin, D. D. O.; Butland, S. L.; Lavallée-Adam, M.; Calzolari, D.; Kay, C.; Yates, J. R.; Hayden, M. R. Curation of the Mammalian Palmitoylome Indicates a Pivotal Role for Palmitoylation in Diseases and Disorders of the Nervous System and Cancers. PLOS Comput. Biol. 2015, 11, e1004405.
(30) Zhang, Z.; Lee, Y.-C.; Kim, S.-J.; Choi, M. S.; Tsai, P.-C.; Xu, Y.; Xiao, Y.-J.; Zhang, P.; Heffer, A.; Mukherjee, A. B. Palmitoyl-Protein Thioesterase-1 Deficiency Mediates the Activation of the Unfolded Protein Response and Neuronal Apoptosis in INCL. Hum. Mol. Genet. 2006, 15, 337-346.
(31) Kim, S.-J.; Zhang, Z.; Hitomi, E.; Lee, Y.-C.; Mukherjee, A. B. Endoplasmic Reticulum Stress-Induced Caspase-4 Activation Mediates Apoptosis and Neurodegeneration in INCL. Hum. Mol. Genet. 2006, 15, 1826-1834.
(32) Sarkar, C.; Chandra, G.; Peng, S.; Zhang, Z.; Liu, A.; Mukherjee, A. B. Neuroprotection and Lifespan Extension in Ppt1−/− Mice by NtBuHA: Therapeutic Implications for INCL. Nat. Neurosci. 2013, 16, 1608-1617.
(33) Levin, S. W.; Baker, E. H.; Zein, W. M.; Zhang, Z.; Quezado, Z. M. N.; Miao, N.; Gropman, A.; Griffin, K. J.; Bianconi, S.; Chandra, G.; Khan, O. I.; Caruso, R. C.; Liu, A.; Mukherjee; A. B. Oral Cysteamine Bitartrate and N-Acetylcysteine for Patients with Infantile Neuronal Ceroid Lipofuscinosis: A Pilot Study. Lancet Neurol. 2014, 13, 777-787.
(34) Runkle, K. B.; Kharbanda, A.; Stypulkowski, E.; Cao, X.; Wang, W.; Garcia, B. A.; Witze, E. S. Inhibition of DHHC20-Mediated EGFR Palmitoylation Creates a Dependence on EGFR Signaling. Mol Cell. 2016, 62, 385-396.
(35) Lazaro, R.; Cros, G.; McNeill J. H.; Serrano, J. Panmedica S. A. Vanadyl Organo-Mineral Compounds, Method for Obtaining Such a Compound, Pharmaceutical Composition Containing this Organo-Mineral Compound. U.S. Pat. No. 5,023,358. Jun. 11, 1991.
(36) American Cancer Society. (Jan. 30, 2019) https://www.cancer.org/cancer/bladder-cancer/about/key-statistics.html.
(37) Kompier, LC.; Lurkin, L; van der Aa, M. N. M.; van Rhijn B. W. G.; van der Kwast. T. F L, Zwarthoff, E. C.; FGFR3, LIRAS, KRAS, NRAS and PIK3CA Mutations in Bladder Cancer and Their Potential as Biomarkers for Surveillance and Therapy, PLoS ONE 2010 5(11): e13821, https://doi.org/10.137/journal.pone.0013821
(38) Wang, Z.; Martin, D.; Molinolo, A. A.; Patel, V.; Iglesias-Bartolome, R.; Degese, M. S.; Vitale-Cross, L.; Chen, Q.; Gutkind, J. S. mTOR co-targeting in cetuximab resistance in head and neck cancers harboring PIK3CA and RAS mutations. J. Natl. Cancer Inst. 2014 Aug. 5; 106(9). pii: dju215. doi: 10.1093/jnci/dju215.
(39) National Institutes of Health. (Mar. 12, 2019) https://ghr.nlm nih.gov/condition/costello-syndrome#statistics.
(40) Cox, A. D.; Fesik, S. W.; Kimmelman, A. C.; Luo, J.; Der. C. J. Drugging the undruggable Ras: mission possible? Nat. Rev. Drug Discov. 2014 November; 13(11): 828-851. doi:10.1038/nrd4389.
(41) American Cancer Society. (Jan. 4, 2018) https://www.cancer.org/cancer/melanoma-skin-cancer/about/key-statistics.html.
(42) American Cancer Society. (Jan. 8, 2019) https://www.cancer.org/cancer/acute-myeloid-leukemia/about/key-statistics.html.
(42) Bacher, U.; Haferlach, T.; Schoch, C.; Kern, W.; Schnittger, S. Implications of NRAS mutations in AML: a study of 2502 patients. Blood 2006 107:3847-3853; doi: https://doi.org/10.1182/blood-2005-08-3522.
(43) American Cancer Society. (Jan. 8, 2019) https://www.cancer.org/cancer/non-small-cell-lung-cancer/about/key-statistics.html.
(44) Runkle, K. B.; Kharbanda, A.; Stypulkowski, E.; Cao, X. J.; Wang, W.; Garcia, B. A.; Witze, E. S. Inhibition of DHHC20-Mediated EGFR Palmitoylation Creates a Dependence on EGFR Signaling. Mol Cell. 2016 May 5; 62(3):385-396. doi: 10.1016/j.molcel.2016.04.003.
(45) BrightFocus Foundation. (Mar. 5, 2019) https://www.brightfocus.org/alzheimers/article/alzheimers-disease-facts-figures.
(46) Bhattacharyya, R.; Barren, C.; Kovacs, D. M. Palmitoylation of Amyloid Precursor Protein Regulates Amyloidogenic Processing in Lipid Rafts. J Neurosci 3 Jul. 2013, 33 (27) 11169-11183; DOI: https://doi.org/10.1523/JNEUROSCI.4704-12.2013.
(47) Andrew, R. J.; Fernandez, C. G.; Stanley, M.; Jiang, H.; Nguyen, P.; Rice, R. C.; Buggia-Prevot, V.; De Rossi, P.; Vetrivel, K. S.; Lamb, R. Parent, A. T.; Holtzman, D. M.; Thinakaran, G. Lack of BACE1 S-palmitoylation reduces amyloid burden and mitigates memory deficits in transgenic mouse models of Alzheimer's disease. Proc. Natl. Acad. Sci. 23 Oct. 2017, 201708568; DOI: 10.1073/pnas.1708568114.
(48) Yang, Y.; Hsu, J. M.; Sun, L.; Chan, L. C.; Li, C. W.; Hsu, J. L.; Wei, Y.; Xia, W.; Hou, J.; Qiu, Y.; Hung, M. C. Palmitoylation stabilizes PD-L1 to promote breast tumor growth. Cell Res. 2019 January; 29(1):83-86. doi: 10.1038/s41422-018-0124-5. Epub 2018 Dec. 4.

What is claimed is:

1. A method of treating a neurological disease in a subject in need thereof, said method comprising administering to said subject a therapeutically effective amount of a compound of formula: 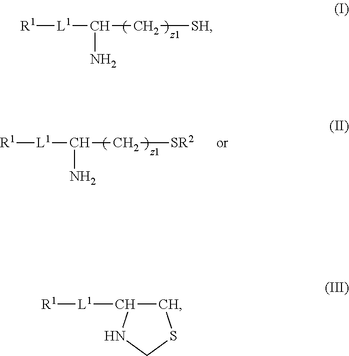
wherein
R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is a thiol protecting group;
R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene; and
z1 is an integer from 0 to 5, thereby treating a neurological disease in said subject.

2. The method of claim 1, wherein said compound is:

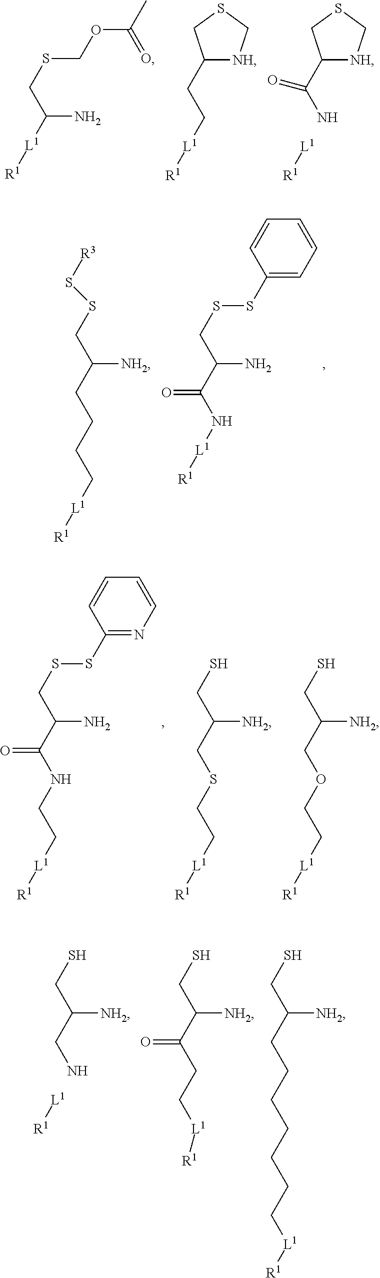

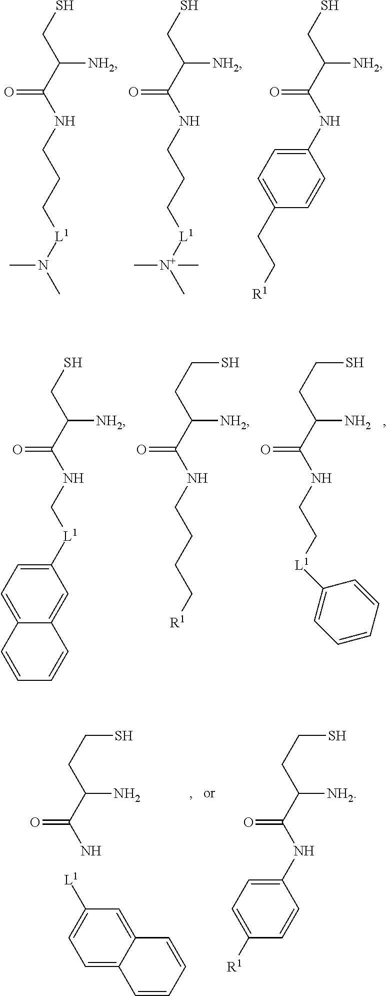

3. A method of treating cancer in a subject in need thereof, said method comprising administering to said subject a therapeutically effective amount of a compound of formula: 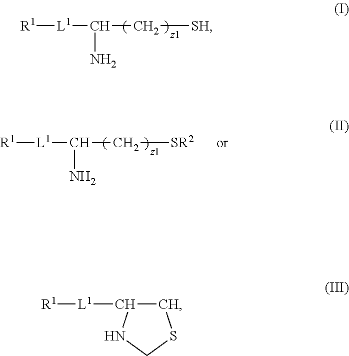
wherein
R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is a thiol protecting group;
R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene; and
z1 is an integer from 0 to 5, thereby treating cancer in said subject.

4. The method of claim 2, wherein said compound is:

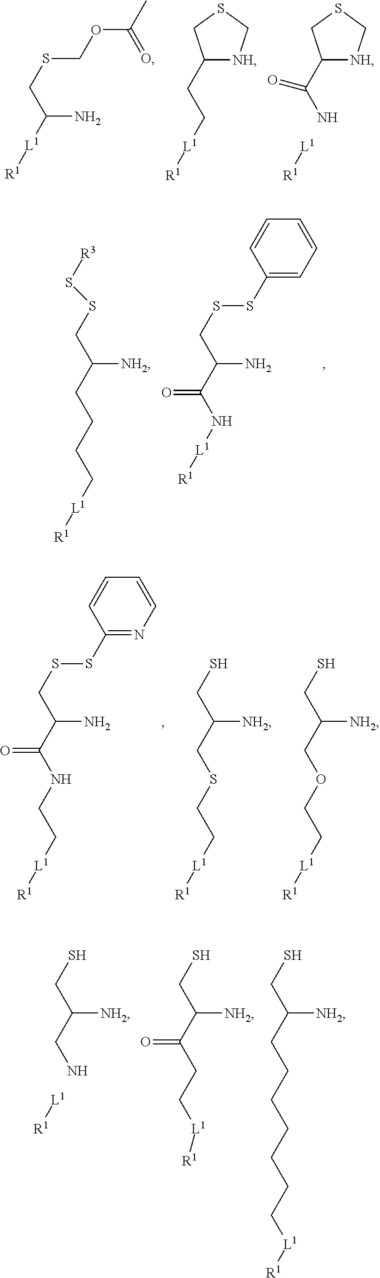

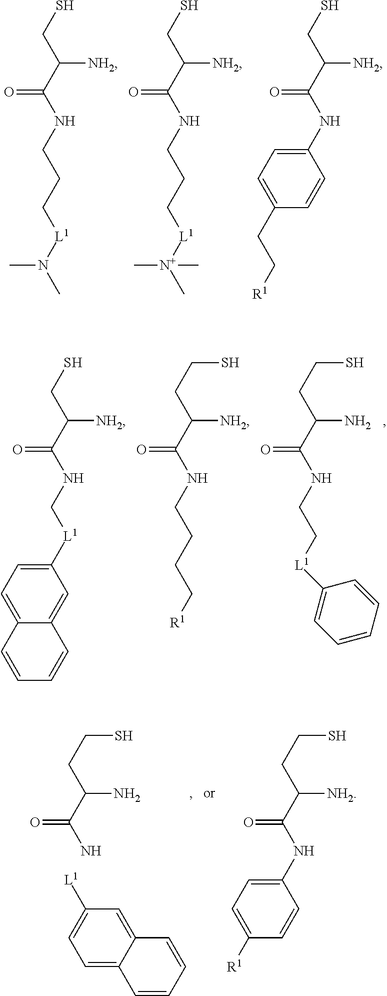

5. A compound of formula: 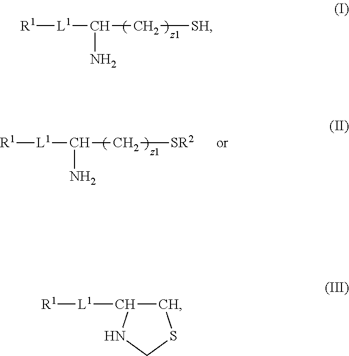
wherein
R¹ is hydrogen, —N(R⁴)(R⁵), —N⁺(R⁴)(R⁵)(R⁶), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is a thiol protecting group;
R⁴, R⁵ and R⁶ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted aryl ene, or substituted or unsubstituted heteroarylene; and
z1 is an integer from 0 to 5.

6. The compound of claim 1, R¹ is —N(R⁴)(R⁵), —N⁺(R⁴)(R⁵)(R⁶), substituted or unsubstituted C₁-C₂₅ alkyl, or substituted or unsubstituted aryl.

7. The compound of claim 5, wherein R¹ is —N(R⁴)(R⁵) and R⁴ and R⁵ are independently unsubstituted C₁-C₁₀ alkyl.

8. The compound of claim 7, wherein R⁴ and R⁵ are independently unsubstituted C₁ alkyl.

9. The compound of claim 6, wherein R¹ is —N⁺(R⁴)(R⁵)(R⁶) and R⁴, R⁵ and R⁶ are independently unsubstituted C₁-C₁₀ alkyl.

10. The compound of claim 9, wherein R⁴, R⁵ and R⁶ are independently unsubstituted C₁ alkyl.

11. The compound of claim 6, wherein R¹ is unsubstituted C₁-C₂₅ alkyl.

12. The compound of claim 5, wherein R¹ is unsubstituted C₁-C₂₅ alkyl.

13. The compound of a of claim 5, wherein R¹ is unsubstituted C₈ alkyl.

14. The compound of of claim 5, wherein, R¹ is R^{1A}-substituted C₁-C₂₅ alkyl,
wherein
R^{1A} is independently hydrogen, —N(R^{4A})(R^{5A}), —N⁺(R^{4A})(R^{5A}×R^{6A}), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{4A}, R^{5A} and R^{6A} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

15. The compound of claim 14, wherein R^{1A} is —N(R^{4A})(R^{5A}) or
—N⁺(R^{4A})(R^{5A})(R^{6A}) and R^{4A}, R^{5A} and R^{6A} are independently substituted or unsubstituted C₁-C₅ alkyl.

16. The compound of claim 14, wherein R^{4A}, R^{5A} and R^{6A} are independently unsubstituted C₁ alkyl.

17. The compound of claim 14, wherein R^{1A} is unsubstituted 5-10-membered aryl.

18. The compound of claim 14, wherein R^{1A} is unsubstituted phenyl or unsubstituted naphthyl.

19. The compound of claim 5, wherein R¹ is substituted or unsubstituted 5-10-membered aryl.

20. The compound of claim 5, wherein R¹ is substituted or unsubstituted phenyl.

21. The compound of claim 5, wherein R¹ is unsubstituted phenyl.

22. The compound of claim 5, wherein R¹ is substituted or unsubstituted naphthyl.

23. The compound of claim 5, wherein R¹ is unsubstituted naphthyl.

24. The compound of claim 5, wherein R¹ is R^{1A}-substituted 5-10-membered aryl, wherein R^{1A} is unsubstituted C₁-C₂₅ alkyl.

25. The compound of claim 24, wherein R¹ is R^{1A}-substituted phenyl and R^{1A} is unsubstituted C₈ alkyl.

26. The compound of claim 5, wherein R² is —SR³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein
R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

27. The compound of claim 5, wherein R² is —SR³ or substituted or unsubstituted heteroalkyl.

28. The compound of claim 5, wherein R² is substituted 2-8 membered heteroalkyl.

29. The compound of claim 5, wherein R² is substituted 4 membered heteroalkyl.

30. The compound of claim 5, wherein R² is

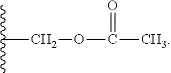 [SMILES: *COC(C)=O]

31. The compound of claim 5, wherein R² is —SR³ and R³ is substituted or unsubstituted C₁-C₅ alkyl.

32. The compound of claim 31, wherein R³ is unsubstituted C₁-C₁₂ alkyl.

33. The compound of claim 5, wherein R² is —SR³ and R³ is substituted or unsubstituted C₅-C₁₀ aryl.

34. The compound of claim 33, wherein R³ is unsubstituted phenyl.

35. The compound of claim 5, wherein R² is —SR³ and R³ is substituted or unsubstituted 5 to 10 membered heteroaryl.

36. The compound of claim 35, wherein R³ is unsubstituted pyridyl.

37. The compound of claim 5, wherein L¹ is a bond, substituted or unsubstituted alkylene or 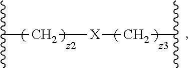
wherein
X is a bond, —S—, —O—, —NH—, —C(O)—NH— or —C(O)—; and
z2 and z3 are independently integers from 0 to 25.

38. The compound of claim 37, wherein X is —C(O)—NH—.

39. The compound of claim 5, wherein z11 or 2.

40. The compound of claim 39, wherein z2 is 0 or 1.

41. The compound of claim 37, wherein z3 is 0, 1, 2 or 4.

42. The compound of claim 37, wherein z3 is an integer from 10-15.

43. The compound of claim 5, wherein L¹ is a bond or unsubstituted C₁-C₈ alkylene.

44. The compound of claim 5, wherein L¹ is unsubstituted C₂ alkylene. or unsubstituted C₄ alkylene.

45. The compound of claim 5, wherein L¹ is unsubstituted C₄ alkylene.

46. A pharmaceutical composition comprising a pharmaceutically acceptable excipient and a compound of claim 5.

47. A method of treating a depalmitoylation-associated disease in a subject in need thereof, said method comprising administering to said subject a therapeutically effective amount of a compound of claim 5, thereby treating a depalmitoylation-associated disease in said subject.

48. The method of claim 47, wherein said depalmitoylation-associated disease is cancer or a neurological disease.

49. The method of claim 47, wherein said depalmitoylation-associated disease is bladder cancer, head and neck cancer, Costello's Syndrome, melanoma, acute myeloid lymphoma (AML), non-small cell lung carcinoma, Alzheimer's disease, infantile neuronal ceroid lipofuscinosis or glioma.

50. A method of depalmitoylating a protein in a cell comprising contacting said cell with an effective amount of a compound of claim 5.

51. The method of claim 50, wherein said protein forms part of the plasma membrane of said cell.

52. The method of claim 50, wherein said protein is HRas, NRas, EGFR, amyloid precursor protein (APP), BACE1, EZH2, PD-L1, flotillin-1, flotillin-2, calnexin, Gα(i), metadherin, CD44 or SNAP25.

53. The method of claim 52, wherein said contacting occurs in vitro or in vivo.

54. The method of claim 53, wherein said cell forms part of an organism.

55. The method of claim 54, wherein said cell forms part of a mammalian subject.

56. The method of claim 55, wherein said mammalian subject suffers from cancer or a neurological disease.

57. A method of treating a depalmitoylation-associated disease in a subject in need thereof, said method comprising administering to said subject a therapeutically effective amount of a depalmitoylating amphiphilic thiol compound, thereby treating a depalmitoylation-associated disease in said subject.

58. The method of claim 57, wherein said depalmitoylating amphiphilic thiol compound is a compound of claim 5.
